(19) United States
(12) Patent Application Publication
(10) Pub. No.: US 2026/0346507 A1
(43) Pub. Date: Nov. 5, 2026

(54) AUTOMATED DISPENSER FOR CASTING HIGH-VISCOSITY SCAFFOLD SOLUTIONS

(71) Applicant: University of Washington, Seattle, WA (US)

(72) Inventors: Miqin ZHANG, Seattle, WA (US); Richard REVIA, Seattle, WA (US); Matthew Michael JAMES, Seattle, WA (US)

(73) Assignee: University of Washington, Seattle, WA (US)

(21) Appl. No.: 19/118,233

(22) PCT Filed: Oct. 10, 2023
(86) PCT No.: PCT/US2023/076482

§ 371 Date: Apr. 3, 2025

Related U.S. Application Data
(60) Provisional application No. 63/415,165, filed on Oct. 11, 2022.

(51) Int. Cl. B29C 39/10; B29C 31/04; B29C 39/24; B29C 39/38; B29L 31/00; C12N 5/00

(52) U.S. Cl. CPC B29C 39/10; B29C 31/042; B29C 39/24; B29C 39/38; C12N 5/0062; B29K 2005/00; B29L 2031/753; C12N 2533/72; C12N 2533/74

(57) ABSTRACT

A viscous material dispenser, including an extruder with an inlet, a barrel, and a nozzle, a pump configured to fit inside the barrel, a tube connected to the inlet, a first valve attached to a first arm of the tube, and a second valve attached to a second arm of the tube, where when the first valve is open and the second valve is closed, the plunger is depressed by the source of pressurized air, causing a viscous material to flow out of the nozzle. Further, a method of dispensing a viscous material using a viscous material dispenser including positioning a substrate underneath the nozzle, filling the barrel of the extruder with a viscous material, and dispensing the viscous material from the nozzle of the extruder and onto the substrate by opening the first valve and closing the second valve.

### CROSS-REFERENCE TO RELATED APPLICATION

This application claims the benefit of U.S. Provisional Application 63/415,165 filed Oct. 11, 2022, the entire disclosure of which is incorporated herein.

### BACKGROUND

In research investigations, biological cells are typically cultured in plastic Petri dishes or flasks, a technique that forces cells to grow upon a rigid, two-dimensional (2D) surface. 2D cell culture methods do not always yield cells that exhibit behavior close to their counterpart cells living in native environments. When cells cultured for study do not recapitulate in vivo phenotypes, results obtained from those cultured cells serve little to no predictability and are often misleading, while animal models are expensive, time consuming, and present ethical dilemmas. To grow cells that express a more physiologically relevant phenotype, three-dimensional (3D) scaffolds have been developed to provide cells with an environment that mimics what the cells would observe in vivo. In contrast to 2D cell culture techniques, 3D cell scaffolds may be engineered to display properties that are like the extracellular matrices (ECMs) preferred by cells. For example, chitosan and alginate, two natural, biocompatible materials were combined in solution to form a polyelectrolyte complex followed by lyophilization to generate a 3D porous chitosan-alginate (CA) scaffold. These polymers share a structure similar to that of glycosaminoglycans which are essential elements of some ECMs. CA scaffolds have been used in tissue engineering for promoting cell penetration, cellular function, and tissue growth, and for culturing cancer cells that yielded a more tumorigenic phenotype in comparison to those cultured in 2D. Similarly, a biodegradable and biocompatible polymer, hyaluronic acid (HA), is one of the major glycosaminoglycan (GAG) components of the ECM found in the brain.

Cells seeded and cultured in 3D scaffolds express a phenotype more like cells grown in vivo than cells cultured in 2D containers, demonstrating a significant advantage of using such engineered scaffolds for tissue engineering research. Efforts in the growth and development of stem cells and organoids for tissue engineering and drug discovery and the search for improved drug therapies targeted against cancer exemplify just two important areas of biomedical applications that can benefit from the superior cell models achieved with 3D cell culture environments. However, a bottleneck in the day-to-day use of 3D scaffolds is that the precursor solutions used to synthesize the scaffolds are typically hand-cast into microplates; manually filling molds with scaffold solution is a time-consuming and labor-intensive process that is prone to producing nonuniform scaffolds across a large number of wells in a microplate. Further, there is a lack of equipment available for handling and dispensing polymer solutions of high viscosity.

While progress has been made in tissue engineering because of the use of 3D scaffolds, a significant bottleneck exists in the production of 3D cell scaffolds. With respect to the large-scale techniques of scaffold synthesis, which include particulate leaching, gas foaming, thermally induced phase separation, and emulsion freeze drying, a general procedure for scaffold production is as follows: (1) creation of solution by dissolving a polymer in an appropriate solvent, (2) casting the polymer solution into a mold (typically the wells of a microplate), and (3) removal of the solvent through evaporation or sublimation. The chokepoint in this process is step two, wherein a polymer solution is cast into a mold by hand. While a large infrastructure exists for conducting high-throughput biological assays using 2D cell culture systems and microplates, the currently existing apparatuses of this infrastructure cannot handle solutions of such high viscosities as are requisite of the polymer solutions that form 3D scaffolds. On the other hand, pneumatically actuated dispensing systems are often employed in the electronics and packaging industries to distribute solder pastes and high-viscosity glues, but these systems are costly and do not meet the needs of tissue engineering researchers requiring full control of dispensing parameters, nor are they designed to comport with the dimensions of microplates.

Further, Adult glioblastoma multiforme (GBM) is one of the most deadly and recalcitrant cancers in the United States. For decades, extensive efforts have been dedicated to cancer drug development, yet the failure rate and the cost of new drug discovery are exceptionally high. One of the major obstacles in new drug development is that the conventional in vitro high-throughput screening (HTS) approach often fails to reliably predict biological responses in vivo. HTS is a widely accepted practice in the Pharmaceutical and Biotech industry to quickly assess a large quantity of compounds in miniaturized in vitro assays. Currently, the traditional 2D in vitro HTS is the most common strategy for novel cancer therapeutics screening. Although relatively cheap and fast, the 2D cell culture cannot closely recapitulate the in vivo 3D tumor tissue microenvironment, which regulates important cell-cell and cell-matrix interactions. Consequently, the effective compounds selected by 2D screening often have a high failure rate in clinical trials and lead to slow and expensive drug development processes.

Compared to cancer cells cultured with 2D monolayers, cancer cells cultured in 3D matrix are able to form self-assembled aggregates, aptly termed “tumor spheroids”, and render cell behaviors similar to those in in vivo tumor tissues. They have been shown to possess more physiologically relevance to in vivo environments, and showed significantly increased cell malignancy, drug resistance and self-renewal abilities. Therefore, much effort has been dedicated to developing 3D tumor spheroid models to improve the efficacy of HTS of cancer drugs.

However, there are obstacles still holding back the translation of such scaffold-based HTS platforms from bench-top research to human clinical trials. First, the polymer solution of chitosan and HA is usually highly viscous and difficult to handle, hampering the large-batch production of CHA scaffolds. Hence, it is vital to establish a scalable manufacturing strategy that can accommodate the 3D CHA scaffold to a HTS platform. Second, human tumors exhibit significant heterogeneity in drug resistance, owing to their inter- and intra-patient genomic and phenotypic diversity. Obtaining precise HTS results can be challenging when testing various types of cancer cells. Thus, it is essential for the scaffold to possess a customizable microenvironment capable of accommodating diverse cancer cell types, consequently optimizing the HTS outcome based on the characteristics of the tumor to be treated.

Accordingly, improved 3D scaffolds, dispensers for 3D scaffolds, and methods of making and using 3D scaffolds are needed.

### SUMMARY

This summary is provided to introduce a selection of concepts in a simplified form that are further described below in the Detailed Description. This summary is not intended to identify key features of the claimed subject matter, nor is it intended to be used as an aid in determining the scope of the claimed subject matter.

In one aspect, described herein is a viscous material dispenser, including an extruder comprising an inlet, a barrel, and a nozzle, a pump configured to fit inside the barrel, a tube connected to the inlet, wherein the tube branches into at least two arms, a first valve attached to a first arm of the tube, wherein the first valve opens and closes to a source of pressure, and a second valve attached to a second arm of the tube, where when the first valve is open and the second valve is closed, the pump is depressed by the source of pressurized air, causing a viscous material to flow out of the nozzle.

In some embodiments, when the first valve is closed and the second valve is open, the viscous material stops flowing out of the nozzle. In some embodiments, the first valve and/or the second valve is selected from a pneumatic valve, a peristaltic valve, a piezoelectric valve, a hydraulic valve, an electromagnetic pump, or a progressive cavity valve. In some embodiments, the source of pressure is pressurized gas. In some embodiments, the source of pressure is pressurized liquid. In some embodiments, the second pneumatic valve opens and closes to the atmosphere.

In some embodiments, the pump is a plunger. In some embodiments, the pump is a progressive cavity pump.

In some embodiments, the viscous material dispenser further comprises a stepper-motor powered moveable stage configured to move a substrate horizontally and vertically in precise step sizes so that one of the wells of the substrate is always directly beneath the nozzle of the extruder when the stepper-motor powered moveable stage is stationary.

In some embodiments, the viscous material dispenser is multiplexed.

In some embodiments, the viscous material dispenser further includes a heater configured to heat the viscous material to a constant temperature to decrease the viscous material's viscosity. In some embodiment, the heater includes a heating pad, and a metal tube surrounding the barrel and mounted to the heating pad. In some embodiments, the metal tube is an aluminum tube. In some embodiments, the heater further comprises a heating jacket surrounding the metal tube. In some embodiments, the heater heats the viscous material to a temperature between 20 °C and 80 °C.

In some embodiments, the first pneumatic valve and the second pneumatic valve are a first solenoid and a second solenoid, respectively. In some embodiments, the extruder is a syringe.

In another aspect, disclosed herein is a method of dispensing a viscous material using a viscous material dispenser described herein, the method including positioning a substrate underneath the nozzle, filling the barrel of the extruder with a viscous material, and dispensing the viscous material from the nozzle of the extruder and onto the substrate by opening the first valve and closing the second valve.

In some embodiments, the method includes stopping the dispensing of the viscous material by closing the first valve and opening the second valve. In some embodiments, the method further includes centrifuging the substrate. In some embodiments, the method includes lyophilizing the substrate to provide a cell scaffold.

In some embodiments, the substrate is a microplate. In some embodiments, the microplate is a 96-well microplate. In some embodiments, the microplate is a 384-well microplate.

In some embodiments, the method further includes actuating a stepper-motor powered moveable stage horizontally and vertically in precise step sizes so that one of the wells of the substrate is always directly beneath the nozzle of the extruder when the stepper-motor powered moveable stage is stationary.

In some embodiments, the method includes heating the viscous material with a heater configured to heat the solution to a constant temperature to decrease the solution viscosity.

In some embodiments, the viscous material dispenser further includes a heater configured to heat the viscous material to a constant temperature to decrease the viscous material's viscosity. In some embodiment, the heater includes a heating pad, and a metal tube surrounding the barrel and mounted to the heating pad. In some embodiments, the metal tube is an aluminum tube. In some embodiments, the heater further comprises a heating jacket surrounding the metal tube. In some embodiments, the heater heats the viscous material to a temperature between 20 °C and 80 °C.

In some embodiments, the first pneumatic valve and the second pneumatic valve are a first solenoid and a second solenoid, respectively.

In some embodiments, the viscous material is a gel. In some embodiments, the viscous material includes chitosan. In some embodiments, the viscous material further includes alginate. In some embodiments, the viscous material includes chitosan-alginate gel. In some embodiments, the viscous material includes chitosan and hyaluronic hybrid gel. In some embodiments, the viscous material includes a polymer solution gel with encapsulated cells. In some embodiments, the viscous material has a viscosity of about 8.9× 10-4 Pa·s to 7000 Pa·s.

In some embodiments, the barrel is filled with one or more layers of two or more gels.

In yet another aspect, disclosed herein is a cell scaffold made by any of the methods disclosed herein, where the cell scaffold has a uniform volume.

In some embodiments, the cell scaffold has a uniform volume of 5% for a 96-well-plate. In some embodiments, the cell scaffold has a uniform volume of 8% for a 384 well-plate. In some embodiments, the cell scaffold is a porous cell scaffold. In some embodiments, the porous cell scaffold has uniform porosity. In some embodiments, the cell scaffold height is uniform among wells of a microplate. In some embodiments, the cell scaffold has a uniform height of 5% for a 96 well plate. In some embodiments, the cell scaffold has a uniform height of 8% for a 384 well plate.

In yet another aspect, disclosed herein is an article of manufacture including a cell scaffold as described herein.

In some embodiments, the article is a cell scaffold for 3D cell culture for fundamental biological studies, 3D in vitro tumor model for drug screening, 3D in vitro disease model for drug discovery, or 3D cell culture for primary and stem cell expansion.

### DESCRIPTION OF THE DRAWINGS

The foregoing aspects and many of the attendant advantages of this disclosure will become more readily appreciated as the same become better understood by reference to the following detailed description, when taken in conjunction with the accompanying drawings, wherein:

FIG. 1A is an example automated scaffold dispenser, in accordance with the present technology;

FIG. 1B is a close up of the extruder of the example automated scaffold dispenser of FIG. 1A, in accordance with the present technology;

FIG. 1C is a circuit diagram of the example automated scaffold dispenser of FIG. 1A, in accordance with the present technology;

FIG. 2A is an example pneumatic housing of an example automated scaffold dispenser, in accordance with the present technology;

FIG. 2B is a close-up view of the example pneumatic housing of FIG. 2A, in accordance with the present technology;

FIG. 2C is an internal view of the example pneumatic housing of FIG. 2A, in accordance with the present technology;

FIG. 3 is an example graphical user interface (GUI) of an example automated scaffold dispenser, in accordance with the present technology;

FIG. 4A is a graph of the viscosity over time of a chitosan alginate (CA) scaffold made with an automated scaffold dispenser, in accordance with the present technology;

FIG. 4B is a graph of the viscosity over time of a chitosan hyaluronic acid (CHA) scaffold made with an automated scaffold dispenser, in accordance with the present technology;

FIGS. 5A-5C show dispensed scaffolds in a 96-well plate, in accordance with the present technology;

FIGS. 6A-6C show dispensed scaffolds in a 384-well plate; in accordance with the present technology;

FIGS. 7A-7F show the scaffold microstructure of dispensed scaffolds in a 96-well plate, in accordance with the present technology;

FIGS. 8A-8F show the scaffold microstructure of dispensed scaffolds in a 384-well plate; in accordance with the present technology;

FIG. 9 is a graph of the pore area of example scaffolds, in accordance with the present technology;

FIG. 10 is a graph of a drug-screening proof of concept using example scaffolds, in accordance with the present technology;

FIGS. 11A-11F are process blocks illustrating a method of making example scaffolds, in accordance with the present technology;

FIGS. 12A-12F show the scaffold microstructure of dispensed scaffolds, in accordance with the present technology;

FIGS. 13A-13C are graphs of pore size distributions of the dispensed scaffolds of FIGS. 12A-12F, in accordance with the present technology;

FIGS. 14A-14C are porous structures of example dispensed scaffolds, in accordance with the present technology;

FIGS. 15A-15B are graphs of porosity of the example dispensed scaffolds of FIGS. 14A-14C, in accordance with the present technology;

FIGS. 16A-16B are graphs of the mechanical properties of the example dispensed scaffolds of FIGS. 14A-14C, in accordance with the present technology;

FIGS. 17A-17I are fluorescent images of live and dead cells of three example scaffolds having different pore sizes, in accordance with the present technology;

FIGS. 18A-18C are graphs of cell proliferation profiles on two dimensional (2D) microplates and in scaffolds of different pore sizes as shown in FIGS. 17A-17I, in accordance with the present technology;

FIGS. 19A-19C are graphs showing gene expression of (GBM) cells on different substrates, in accordance with the present technology;

FIGS. 20A-20C are graphs of drug responses of GBM cells on different substrates, in accordance with the present technology; and

FIGS. 21A-21B are graphs characterizing the quality of the scaffolds, in accordance with the present technology.

### DETAILED DESCRIPTION

While illustrative embodiments have been illustrated and described, it will be appreciated that various changes can be made therein without departing from the spirit and scope of the disclosure.

In one aspect, described herein is a viscous material dispenser, including an extruder comprising an inlet, a barrel, and a nozzle, a pump configured to fit inside the barrel, a tube connected to the inlet, where the tube branches into at least two arms, a first pneumatic valve attached to a first arm of the tube, wherein the first valve opens and closes to a source of pressure, and a second pneumatic valve attached to a second arm of the tube, where when the first pneumatic valve is open and the second pneumatic valve is closed, the pump is depressed by the source of pressurized air, causing a viscous material to flow out of the nozzle. In another aspect, disclosed herein is a method of dispensing a viscous material using a viscous material dispenser according to the disclosure herein, the method including positioning a substrate underneath the nozzle, filling the barrel of the extruder with a viscous material, and dispensing the viscous material from the nozzle of the extruder and onto the substrate by opening the first pneumatic valve and closing the second pneumatic valve. In yet another aspect, disclosed herein is a cell scaffold made by any of the methods disclosed herein, where the cell scaffold has a uniform volume. In yet another aspect, disclosed herein is an article of manufacture including a cell scaffold as described herein.

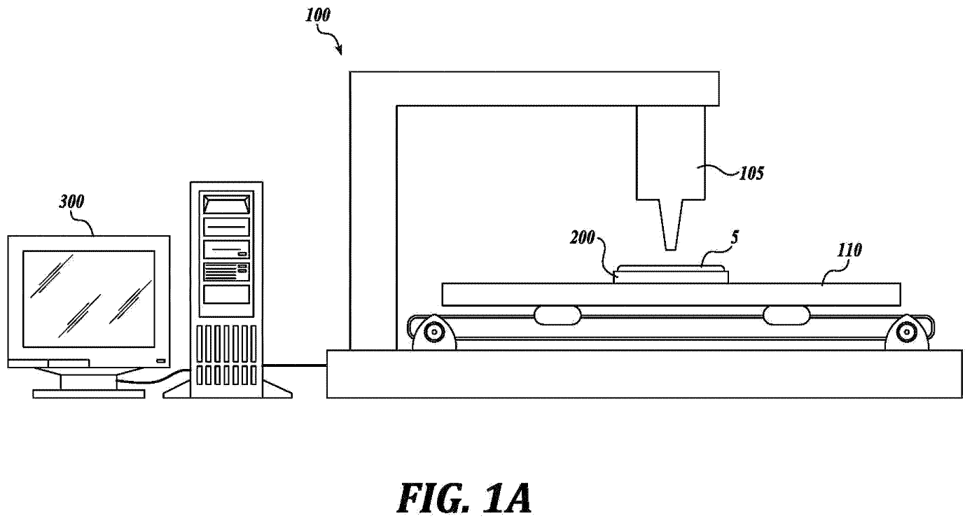

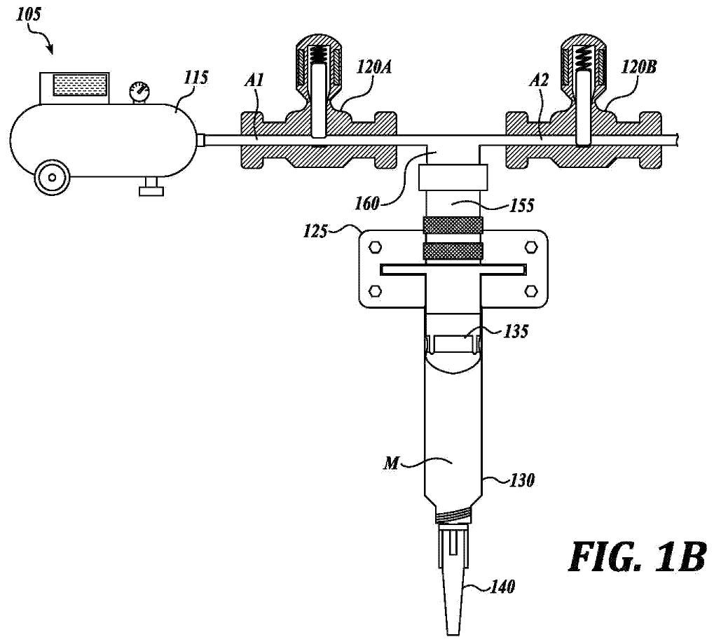

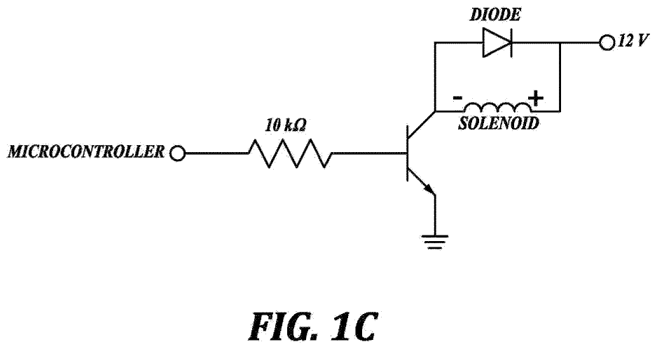

FIG. 1A is an example automated scaffold (or viscous material) dispenser 100, in accordance with the present technology. In some embodiments, the automated scaffold dispenser 100 (or apparatus) is based on a stepper-motor powered movable stage 110 and a custom extruder 105 capable of distributing high-viscosity materials (or scaffold materials) onto a microplate 200. In some embodiments, the automated scaffold dispenser 100 is communicatively or physically coupled to a computer 300.

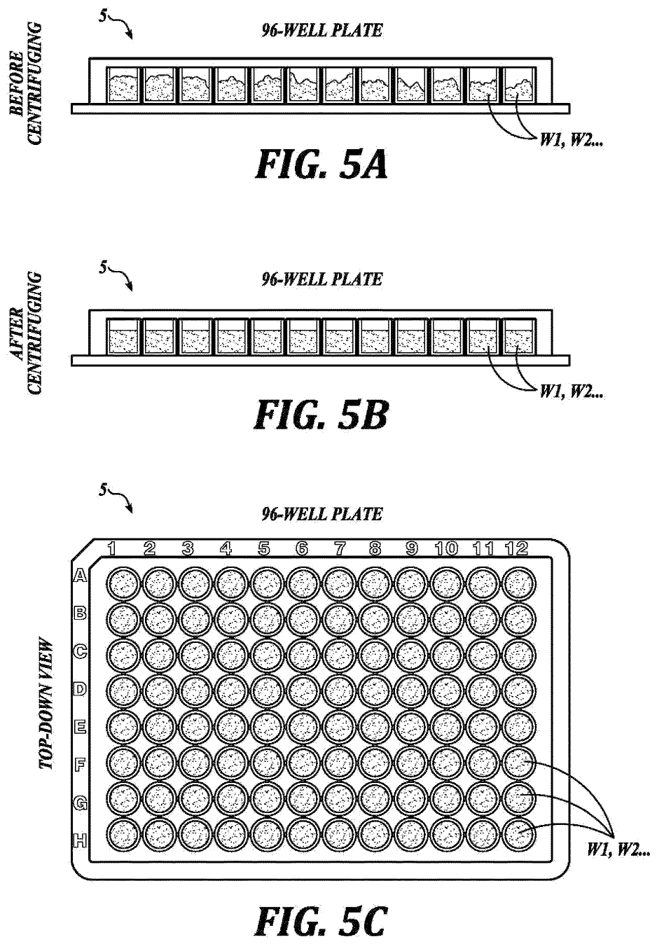

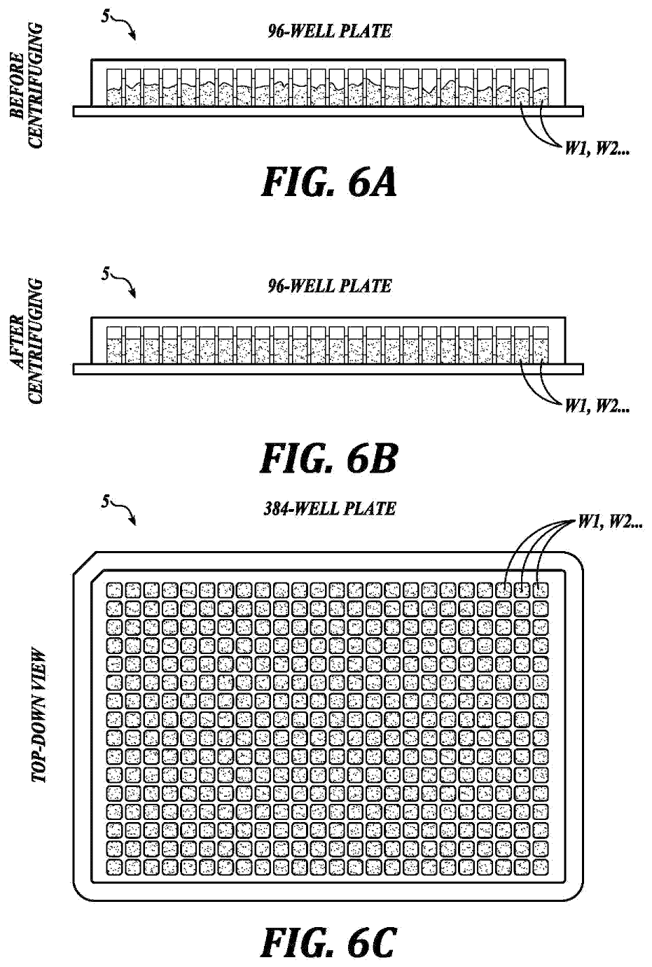

In operation, the movable stage 110 is configured to move the microplate 200 underneath the extruder 105. In some embodiments, the stepper-powered moveable stage 110 is configured to move a substrate (or microplate) 200 horizontally and/or vertically in precise step sizes so that one of the wells (such as wells W1, W2 of FIGS. 5A-5C and 6A-6C) of the substrate 200 is always directly beneath a nozzle (such as nozzle 140 of FIG. 1B) of the extruder 105 when the movable stage 110 is stationary. In some embodiments, the computer 300 is configured to direct the automated scaffold dispenser 100 to dispense scaffold materials onto the microplate 200. In some embodiments, the viscous material dispenser 100 is multiplexed, that is it has multiple extruders 105, configured to fill multiple wells of substrate 200 at a time.

FIG. 1B is a close up of the extruder 105 of the example automated scaffold dispenser 100 of FIG. 1A, in accordance with the present technology. In some embodiments, the extruder 105 may be one of a plurality of extruders attached to the scaffold dispenser 100 of FIG. 1A. In some embodiments, the extruder 105 is a pneumatic extruder. In some embodiments, the extruder 105 includes an external source of pressurized gas 115, a first pneumatic valve 120A, and a second pneumatic valve 120B. In some embodiments, the pneumatic valves 120A, 120B are a first solenoid and a second solenoid, respectively. In some embodiments, the extruder 105 includes a syringe 130, a plunger 135 (also referred to herein as a pump) located inside the syringe, an inlet 155, and a nozzle 140. In some embodiments, the extruder 105 is attached to an automated scaffold dispenser (such as automated scaffold dispenser 100 of FIG. 1A) with a mounting bracket 125. In some embodiments, the pump (or plunger) 135 is configured to fit inside the barrel 130. In some embodiments, the pump 135 is a plunger. In some embodiments, the pump 135 is a progressive cavity pump. In some embodiments, the extruder is a syringe.

In some embodiments, a tube 160 is connected to the inlet 155. In some embodiments, the tube 160 branches into at least two arms A1, A2. In some embodiments, the first pneumatic valve 120A attached to a first arm A1 of the tube 160. In some embodiments, the external source of pressurized gas 115 is an air compressor, and is configured to depress the plunger 135 of the syringe 130 filled with scaffold solution (or scaffold material) M.

In operation, management of the applied pressure is achieved with a pressure regulator (not pictured in FIG. 1B) and two computer-controlled (such as with the computer 300 of FIG. 1A) valves 120A, 120B. In FIG. 1B, the first valve 120A is shown open, while the second valve 120B is shown closed. In some embodiments, the first valve 120A opens and closes to the source of pressure 115. In some embodiments, the first valve 120A and/or the second valve 120B is selected from a pneumatic valve, a peristaltic valve, a piezoelectric valve, a hydraulic valve, an electromagnetic pump, a solenoid, or a progressive cavity valve. In some embodiments, the source of pressure 115 is pressurized air. In some embodiments, the source of pressure 115 is pressurized liquid. In some embodiments, a second valve 120B is attached to a second arm A2 of the tube 160. In some embodiments, when the first valve 120A is open and the second valve 120B is closed, the pump 135 is depressed by the source of pressurized air 115, causing viscous material M to flow out of the nozzle 140. In some embodiments, the second pneumatic valve 120B opens and closes to the atmosphere. In one aspect, disclosed herein is a method of dispensing a viscous material using viscous material dispenser described herein. In some embodiments, the method includes positioning the substrate 200 underneath the nozzle 140, filling the barrel 155 of the extruder 105 with a viscous material M, and dispensing the viscous material M from the nozzle 140 of the extruder 105 and onto the substrate 200 by opening the first valve 120A and closing the second valve 120B. In some embodiments, the method further includes stopping the dispensing of the viscous material by closing the first pneumatic valve 120A and opening the second valve 120B.

FIG. 1C is a circuit diagram of the example automated scaffold dispenser of FIG. 1A, in accordance with the present technology. Specifically, FIG. 1C shows a solenoid valve control circuit. The circuit diagram includes a microcontroller, a 10 kΩ resistor, a diode, a solenoid (such as valves 120A, 120B), and a 12 V voltage source. In operation a computer (such as computer 300) is configured to control the solenoid to open or close.

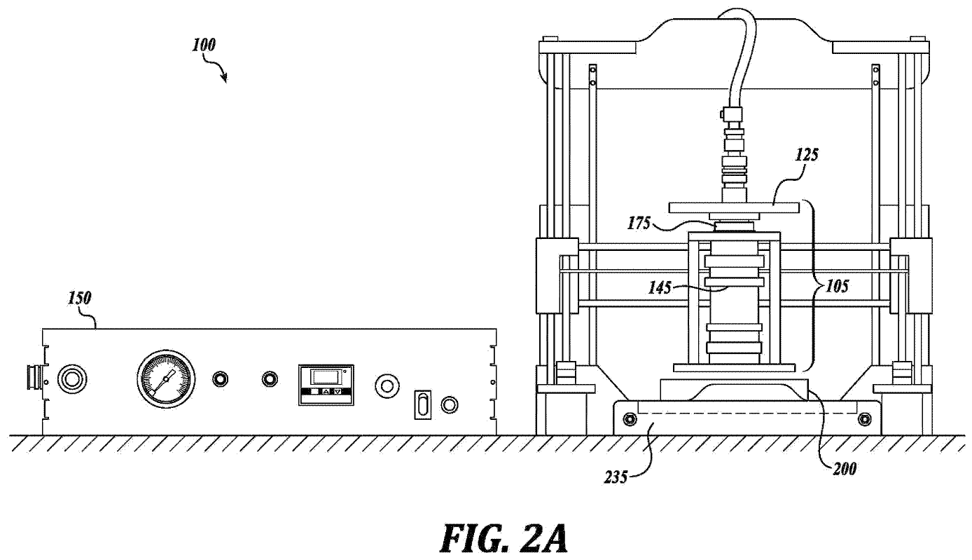

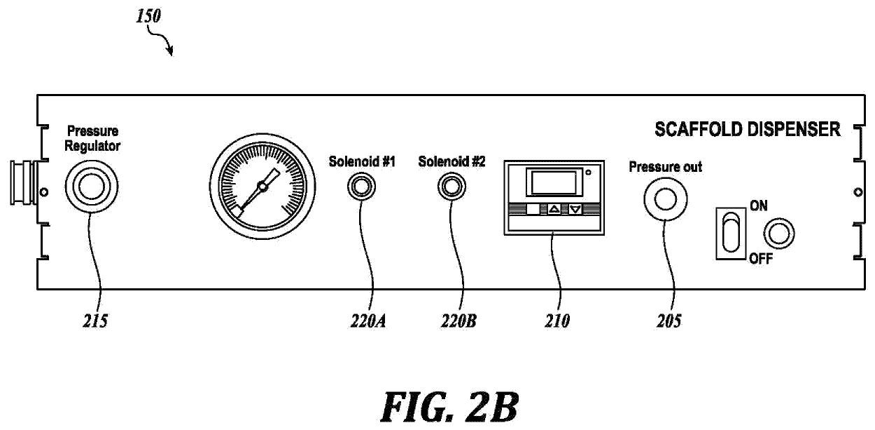

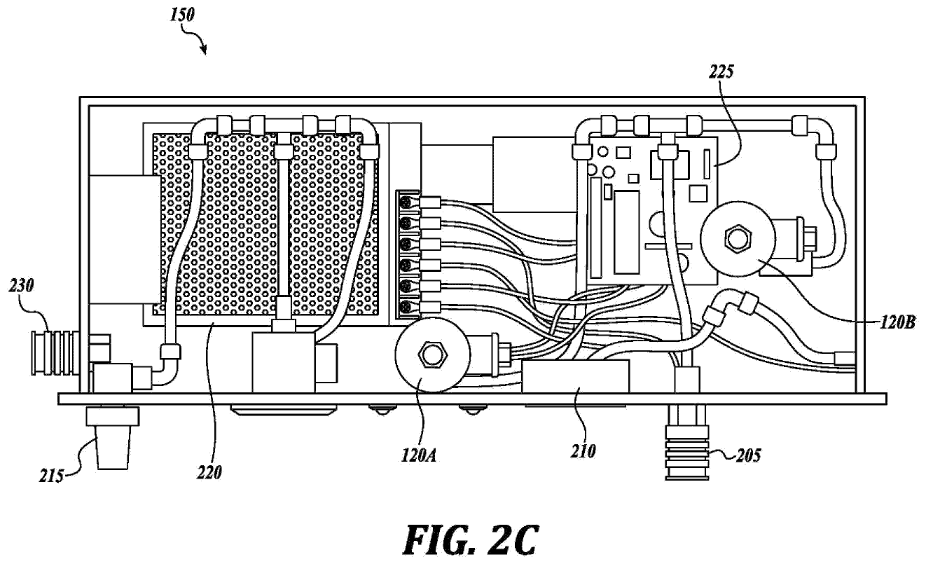

FIG. 2A is an example housing 150 of an example automated scaffold dispenser 100, in accordance with the present technology. In some embodiments, the automated scaffold dispenser 100 includes a housing 150, which contains a first valve and a second valve (such as first valve 120A, and the second valve 120B of FIG. 1B). In some embodiments, the automated scaffold dispenser 100 includes an extruder 105. In some embodiments, the automated scaffold dispenser 100 includes a moving plate 110, and a mounting bracket 125.

In some embodiments, such as the one illustrated in FIGS. 2A-2C, the scaffold dispensing apparatus 100 further includes a heater (as shown in FIG. 2C) configured to heat a viscous material (such as scaffold material M of FIG. 1B) to a constant temperature to decrease the viscous material's velocity. In some embodiments, the heater may include a metal tube 175 that surrounds the syringe barrel 130. In some embodiments, the metal tube 175 is an aluminum tube. In some embodiments, the heater further includes a heating jacket 145 configured to surround the metal tube 175. In some embodiments, the heater includes or consists of a heating pad 235. In this manner, the heater may be a metal tube 175, a heating jacket 145, a heating pad, or a combination thereof. In some embodiments, the heater is an 80 W heating pad 235. In some embodiments, the heater heats the viscous material to a temperature between 20 °C and 80 °C.

FIG. 2B is a close-up view of the example housing 150 of FIG. 2A, in accordance with the present technology. In some embodiments, the housing 150 includes a pressure regulator 215, a first solenoid indicator 220A, a second solenoid indicator 220B, a heater 210, and an air out port 205. In some embodiments, the temperature of the heater 210 is controlled via a thermostat (not pictured) residing in the housing unit 150. The syringe barrel 130 may be filled with scaffold solution and may be loaded inside the aluminum tube. In operation, when dispensing, the scaffold solution can be heated to a constant temperature in order to decrease the solution viscosity and facilitate distribution of high-viscosity fluids. In some embodiments, the heater 210 heats the viscous material to a temperature between 20 °C and 80 °C. The user can opt for non-heated dispensing when the fluid under study has low viscosity or if dispensing at medium viscosities. The amount of pressure applied during dispensing can be set using the adjustable pressure regulator 215. The first solenoid indicator 220A and the second solenoid indicator 220B may visually display whether either the first solenoid or the second solenoid is open or closed, functioning, or the like. In some embodiments, the first solenoid indicator 220A and the second solenoid indicator 220B are LEDs. The air out port 205 may be coupled to the automatic scaffold dispenser and supply the pressurized air to the syringe, to dispense the scaffold materials.

FIG. 2C is an internal view of the example housing 150 of FIG. 2A, in accordance with the present technology. In some embodiments, the housing 150 is a pneumatic housing. The pneumatic housing unit 150 may include a pressure regulator 215, a power supply 220, a first valve (or solenoid) 120A, a second valve (or solenoid) 120B, a microcontroller (or MCU) 225, a heater 210, and an air out port 205. Pressurized gas is introduced to the housing (or pneumatic system) 150 through the quick connect fitting 230 on the extreme left and is attached to the syringe barrel via the air out port 205 connection.

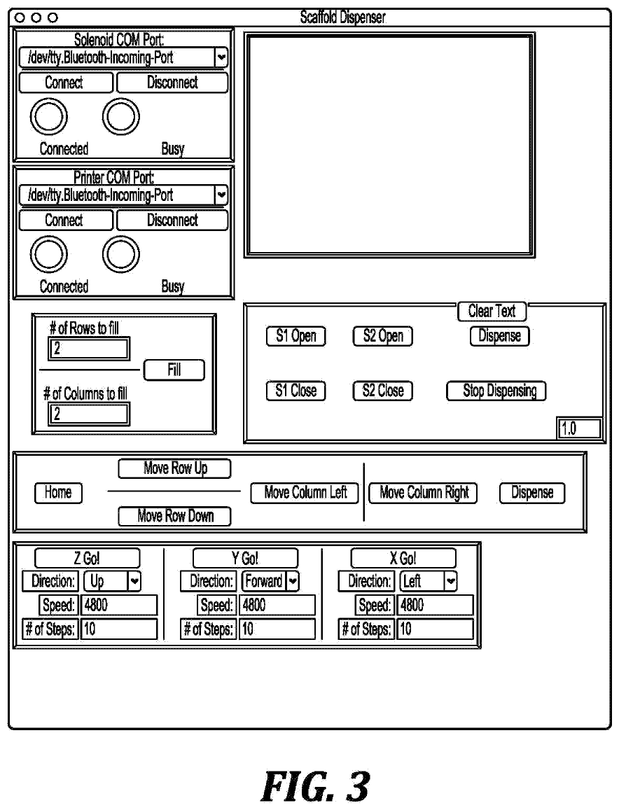

FIG. 3 is an example graphical user interface (GUI) of an example automated scaffold dispenser, in accordance with the present technology. Control of the scaffold dispensing system may be operated through a custom-designed graphical user interface (GUI); a screenshot of an example GUI is displayed in FIG. 3. Through this program, a user can connect to the 3D printer to control the location of the microplate and scaffold extruder. The user can separately connect to the microcontroller unit (MCU) that controls the solenoids. The user can select the type of well plate they wish to dispense into (e.g., 24, 48, 96, or 384). The number of steps the stepper motors required is the number required to translate the microplate the exact distance needed to position the scaffold extruder tip over the center of an adjacent well has been predetermined and hard coded into the software. Similarly, the user can adjust the dwell time, which is the amount of time Solenoid 1 is open. Longer dwell times will apply air pressure to the scaffold solution for a greater duration. The appropriate amount of dwell time corresponds to the volume of the wells for a given microplate (i.e., larger well volume can accommodate more scaffold material, so pressure must be applied for a longer period of time in order to dispense more scaffold solution). Furthermore, the optimal dwell time is unique to a particular scaffold solution mixture, with higher viscosity polymer solutions typically requiring longer dwell times. However, the dwell time can be shortened by lowering the viscosity of the scaffold solution to be dispensed by increasing the temperature of the heating jacked surrounding the syringe barrel. Users may also input the number of rows and columns they wish to fill so that they can either fill each well of a microplate or just a small subset of wells.

### EXAMPLES

Disclosed herein is an automated scaffold dispensing system for rapid, reliable, and homogeneous creation of scaffolds in well-plate formats. In some embodiments, the pneumatically actuated dispensing system can evenly distribute high-viscosity chitosan and chitosan-alginate polymer solutions into 96- and 384-well plates to yield highly uniform three-dimensional scaffolds after lyophilization, as analyzed through scanning electron microscopy and pore size analysis. After scaffolds are created in a 384-well plate, described herein is a proof-of-concept demonstration of high-throughput drug screening by culturing glioblastoma cells in the scaffolds and exposing them to temozolomide, a standard-of-care chemotherapeutic agent for GBM treatment. This work introduces a new device that can greatly enhance the speed with which tissue engineering research may be achieved using three-dimensional cell scaffolds.

### Example #1: Creation and Testing of Chitosan Alginate Gels for Drug Screening

In some embodiments, the viscous material may be a gel. In some embodiments, the viscous material may have a viscosity of about 8.9×10⁻⁴ Pa·s to 7000 Pa·s. To demonstrate the ability of the scaffold dispenser to uniformly distribute scaffold materials into microplates, two exemplary polymer solutions (chitosan-alginate, and CHA) were chosen, but in other embodiments, the viscous material may be any viscous material, such as polymer solutions, and polymer solutions encapsulated with cells, and gels. Chitosan and alginate are two widely used biocompatible materials that achieve excellent cell models when they are used as the substrate for cell culture. Chitosan, a derivative of chitin, is a natural cationic polysaccharide derived from shrimp shells; alginate is a natural anionic polysaccharide derived from brown algae. Both materials have been shown to be biodegradable and induce a negligible immune response when exposed to living systems. Additionally, both polymers share a structure that is similar to glycosaminoglycans, which are essential components of some ECMs. When used as a 3D cell scaffold, chitosan promotes cell adhesion, proliferation, and differentiation due to its hydrophilicity. CA scaffolds were synthesized in previous studies for stem cell renewal due to their proxy structures of GAG and for tumor modeling due to its ability to mimic the tumor microenvironment and improve the tumorigenic potential of cultured cells. Another biocompatible and biodegradable polymer is HA, a natural anionic polymer found in synovial fluid, cartilage, and skin; it is also one of the major glycosaminoglycan components in the brain's ECM. CHA scaffolds were synthesized in previous studies to serve as a mimic of the glioblastoma tumor microenvironment and to promote cartilage regeneration. Previous studies using chitosan-based 3D scaffolds for cell culture have yielded promising results that show the ability of these scaffolds to culture cancer cells that exhibit a more malignant and drug resistant phenotype compared to cancer cells cultured in Petri dishes.

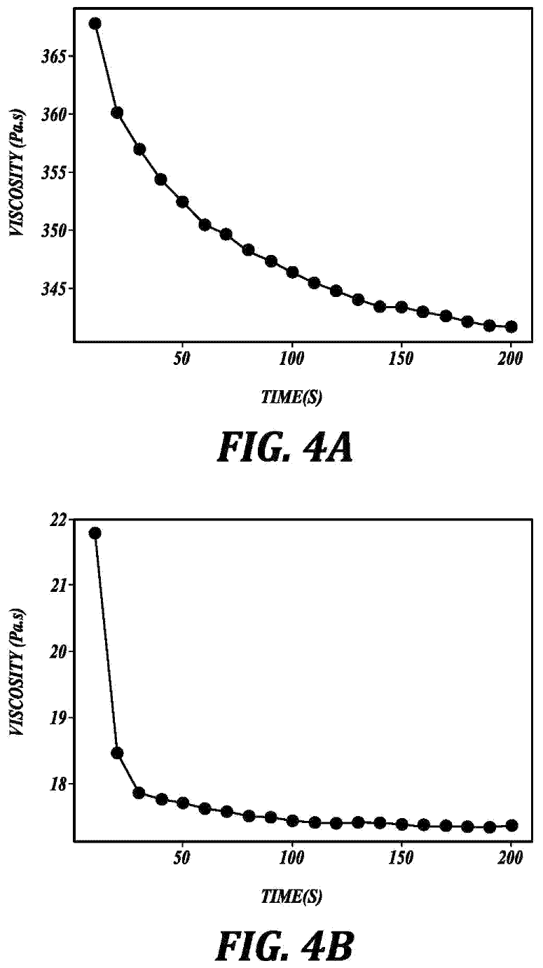

FIG. 4A is a graph of the viscosity over time of a chitosan-alginate (CA) scaffold made with an automated scaffold dispenser, in accordance with the present technology. On the vertical axis is the viscosity in Pa·s. On the horizontal axis is the time in seconds.

FIG. 4B is a graph of the viscosity over time of a chitosan hyaluronic acid (CHA) scaffold made with an automated scaffold dispenser, in accordance with the present technology. On the vertical axis is the viscosity in Pa·s. On the horizontal axis is the time in seconds.

CA and CHA scaffolds were synthesized at polymer concentrations of 2, 4, and 8 wt %. FIGS. 4A-4B displays rheological assessments of 4 wt % CA and CHA scaffolds, respectively, performed at 25 °C. For reference, water has a viscosity of about 8.9×10⁻⁴ Pa·s at 25 °C, but 4 wt % CA and CHA exhibit a viscosity of at least four orders of magnitude greater at about 3.7×10² Pa·s for CA and 2.1×10¹ Pa·s for CHA. The large values of viscosity attendant to the solutions used to generate 3D scaffolds are why hand-casting is such a laborious and time-consuming process, and why specialized dispensing equipment is required for handling such solutions. Viscosity was plotted against time, and FIGS. 4A-4B show both scaffold solutions have a shear thinning characteristic.

FIGS. 5A-5C show dispensed scaffolds in a 96-well plate (or substrate) 200, in accordance with the present technology. In some embodiments, the substrate 200 includes a plurality of wells W1, W2 in which scaffold solution (or material) is dispensed. FIGS. 5A-5C displays photographs of 2 wt % CA scaffolds dispensed in a 96-well plate. The dispensing time for this scaffold solution in the 96-well microplates was roughly 4 min.

FIGS. 6A-6C show dispensed scaffolds in a 384-well plate (or substrate) 200, in accordance with the present technology. In some embodiments, the substrate 200 includes a plurality of wells W1, W2 in which scaffold solution (or material) is dispensed. FIGS. 6A-6C are photographs of 2 wt % CA scaffolds dispensed in a 384-well plate. The dispensing time for this scaffold solution in the 384-well microplates was roughly 15 min.

Table 1 shows the various parameters used during this experiment. FIGS. 5A and 6A depicts the microplates immediately after automated dispensing. Note that, after dispensing, the height of the scaffold solution in the wells is not uniform; in order to address this issue, each microplate was centrifuged for 1 min at 1500 rpm immediately after dispensing to allow the scaffold solution to settle to the bottom of the well and form a homogeneous shape from well-to-well as shown in FIGS. 5B-5C and 6B-6C. In some embodiments, the dispenser may be filled with one or more layers of two or more gels, to create a multilayered scaffold. FIGS. 5C and 6C show top-down views of fully populated 96- and 384-well microplates.

TABLE 1
Parameters used for automated casting of CA and CHA scaffold material

| | CA | | | | | | CHA | | | | | |
|---|---|---|---|---|---|---|---|---|---|---|---|---|
| | Well Plate | | | | | | | | | | | |
| | 96 | | | 384 | | | 96 | | | 384 | | |
| | Weight Percent (%) | | | | | | | | | | | |
| | 2 | 4 | 8 | 2 | 4 | 8 | 2 | 4 | 8 | 2 | 4 | 8 |
| Pressure (kPa) | 15 | 30 | 60 | 15 | 30 | 60 | 10 | 20 | 40 | 10 | 20 | 40 |
| Dwell Time (ms) | 120 | 120 | 120 | 120 | 120 | 120 | 100 | 100 | 100 | 100 | 100 | 100 |
| Total Time (min) | 4 | 4 | 4 | 15 | 15 | 15 | 4 | 4 | 4 | 15 | 15 | 15 |

Dispensed and lyophilized CA and CHA scaffolds were analyzed by SEM to examine their pore size, morphology, interconnectivity, and uniformity. The main purpose of imaging was to confirm the existence of pores within the microstructure of the scaffolds. Scaffold porosity may be altered and optimized by tuning processing parameters such as solution viscosity, polymer concentrations, freezing rates, freezing temperatures, and acetic acid concentrations.

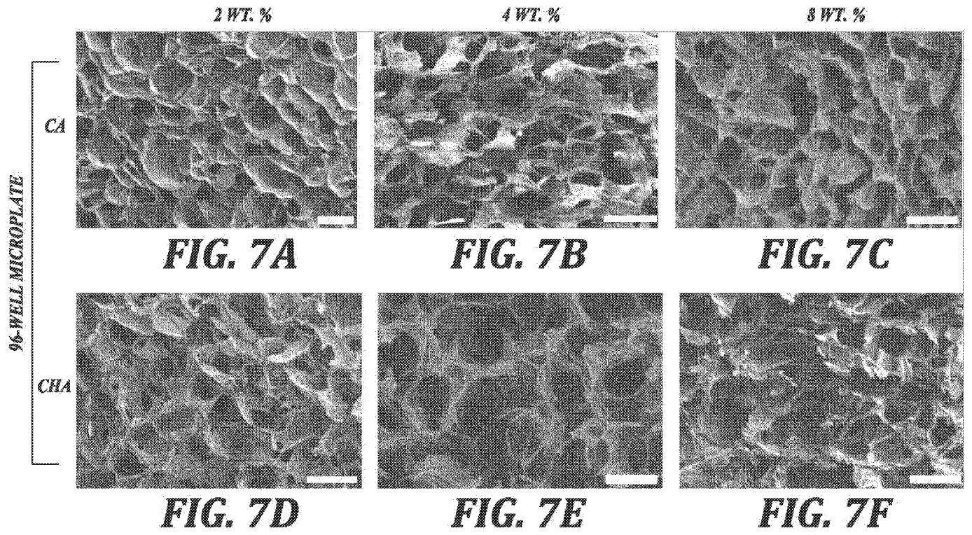

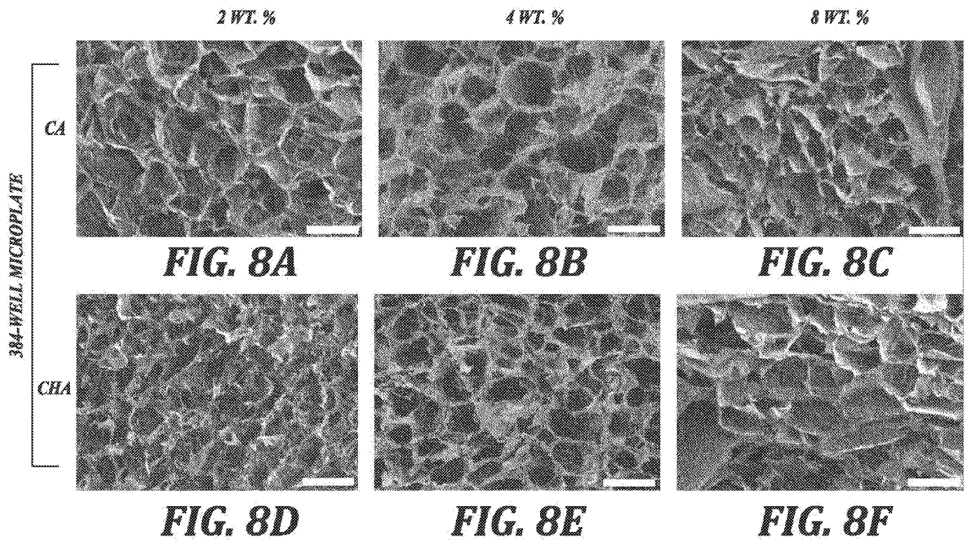

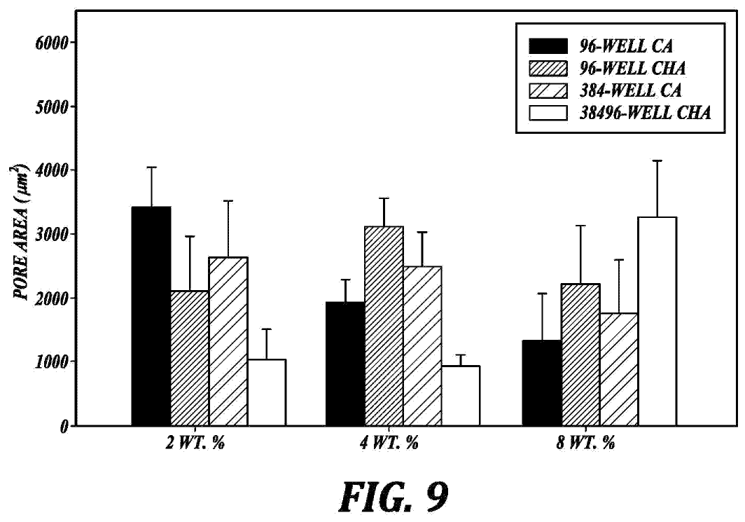

FIGS. 7A-7F show the scaffold microstructure of dispensed scaffolds in a 96-well plate, in accordance with the present technology. FIGS. 8A-8F show the scaffold microstructure of dispensed scaffolds in a 384-well plate, in accordance with the present technology. FIGS. 7A-7F and 8A-8F are evaluations of scaffold microstructure in both 96- and 384-well plates. Cross-sectional SEM images of CA and CHA scaffolds of 2, 4, and 8 wt. % polymer cast in 96- and 384-well plates. Scale bars represent 100 μm. Despite a high degree of variability between the pores sizes of the scaffolds, as shown in the SEM images of FIGS. 7A-7F and FIGS. 8A-8F, and the pore size measurements reported in FIG. 9, pores were successfully generated in 2, 4, and 8 wt. % CA and CHA scaffolds dispensed in both 96- and 384-well microplates. Dispensing of the scaffold solutions was non-trivial because their viscosities change with varying shear rates (i.e., they are non-Newtonian fluids). Rheological measurements of 4 wt. % CA and CHA solutions determined these polymer solutions to exhibit pseudoplastic thixotropic behavior where their viscosities decrease over time under a constant shear rate, as shown in FIGS. 4A-4B. Since viscosity is not independent of time when a constant force is applied, flow of the scaffold solution out of its container during dispensing is not uniform. However, the changing viscosity eventually reaches a plateau during prolonged exposure to force. To reach this region of relatively constant viscosity, several “dummy” extrusions of scaffold are applied prior to dispensing into a microplate in order to obtain a scaffold solution of consistent viscosity, thus achieving a uniform volume of solutions dispensed in each well of a microplate.

The large amount of variability in pore size and morphology exhibited by the CA and CHA scaffolds herein may be attributed to insufficient blending during synthesis of scaffold solutions, resulting in a heterogeneous dispensing solution and subsequent creation of rough surfaces and non-uniform ice crystal growth during lyophilization. Additionally, the elongated pores evident in SEM images of a subset of the created scaffolds may be a result of adding more than 1 wt. % of acetic acid to the scaffold solution, causing the solution viscosity to increase significantly and thereby result in a degradation of the desired pore structure. The large increase of solution viscosity due to higher amounts of acetic acid causes the diffusion rate of the scaffold polymer to decrease. When mobility of a polymer is low during crystallization and recrystallization of ice crystals, polymer dendrites cannot fully migrate to form the walls of the scaffold's pores. Consequently, large irregular pores are formed when solution viscosity is high. Pore irregularity cannot be attributed to the scaffold dispensing technique itself because compressed air would only introduce gas bubbles within the solution, which is degassed during centrifugation.

FIG. 9 is a graph of the pore area of example scaffolds, in accordance with the present technology. Pore area of CA and CHA scaffolds. The mean pore area of 2, 4, and 8 wt. % CA and CHA scaffolds dispensed in both 96- and 384-well plates. Data are displayed as the average with the error bars indicating the standard deviation (n≥60).

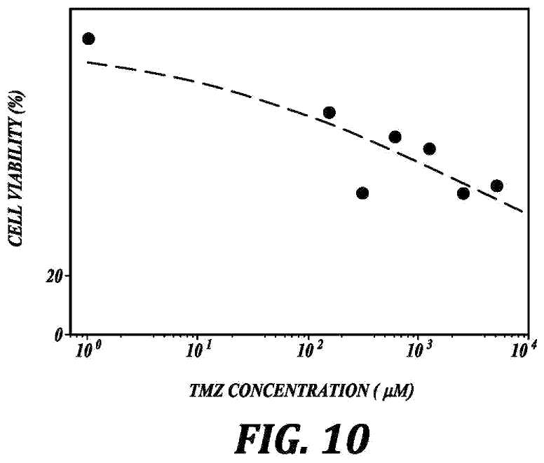

CA scaffolds (4 wt. %) were dispensed in a 384-well microplate and used for in vitro drug screening to evaluate the cytotoxicity of the standard-of-care chemotherapeutic TMZ against glioma cells (U-118 MG). Cell viability of U-118 MG cells cultured on the CA scaffolds was measured as a function of TMZ concentration, and results are shown in FIG. 10.

All chemicals were purchased from Sigma-Aldrich (St. Louis, MO) unless otherwise specified. Chitosan (85% deacetylated, medium molecular weight), alginic acid sodium salt (from brown seaweed, MW=80-120 kDa), and hyaluronic acid sodium salt (from Streptococcus equi) were used as received. Dulbecco's modified Eagle media (DMEM), penicillin streptomycin (Pen Strep), and Dulbecco's phosphate-buffered saline (DPBS) were purchased from Gibco (Gaithersburg, MD). AlamarBlue reagent was purchased from Invitrogen (Carlsbad, CA). Fetal bovine serum (FBS) was purchased from Atlanta Biologicals (Atlanta, GA).

A commercial 3D printer (P802NA, Shenzhen Zonestar Innovation Technology Co, Shenzhen, China) was cannibalized and modified by removing its 3D filament extruder and replacing it with a custom-made pneumatic scaffold dispensing nozzle. The pneumatic scaffold dispensing system consists of an air pressure supply (provided through the laboratory gas infrastructure), a pressure regulator (R25-02b, Parker Watts, Cleveland, OH), two 12 V DC solenoid valves (P0558, BACOENG), a solenoid control circuit, a syringe barrel holder (custom-made from quarter-inch thick clear acrylic sheets), and an 80 W heating pad (SHS0024, Tempco, Wood Dale, IL) controlled by a thermostat (HJ Garden XH-W3002). A 12 V DC supply (PMT-12V150W1aa, Delta Electronics, Taipei, Taiwan) provides power to the solenoids. Control of the solenoids is achieved using low voltage DC signals provided by an ATmega2560 MCU (Microchip Technologies, Chandler, AZ) to bias an n-channel MOSFET (IRF630, STMicroelectronics, Geneva, Switzerland) that acts as a switch. A flyback diode (IN5400RLG, ON Semiconductor, Phoenix, AZ) was placed in parallel with each solenoid.

To position each well of the microplate to be filled underneath the scaffold dispensing nozzle, a GUI was developed in the Python programming language (Python Software Foundation, Wilmington, DE). The GUI program controls both the 3D printer's stepper motors and the scaffold dispenser's solenoid valves to move the microplate and provide pressure to drive out scaffold solution through a nozzle.

Three CA scaffold solutions (2, 4, and 8 wt. %) were prepared by slowly dissolving 2, 4, and 8 g of alginic acid sodium salt in 199 g of deionized water. The solution was mixed in a planetary centrifugal mixer (Thinky ARM-300, Thinky USA, Laguna Hills, CA) at 2000 rpm for 3 min to dissolve residual clumps of polymer. Chitosan powder (2, 4, and 8 g) was introduced to the solution once the alginic acid sodium salt was fully dissolved. The CA solution was mixed again in the planetary centrifugal mixer at 2000 rpm for 3 min to evenly distribute the chitosan powder within the solution. Acetic acid was added dropwise to make a 1 wt. % acetic acid solution. The solution was mixed in the planetary centrifugal mixer at 2000 rpm for 5 min. After dissolution of the chitosan powder, the polymer mixture was blended twice for 5 min to homogenize the polymer solution. The mixture was cooled in an ice bath after blending steps to remove excess heat from within the solution.

Three CHA solutions (2, 4, and 8 wt. %) were prepared by separately dissolving 2, 4, and 8 g of chitosan powder and 1 g of HA sodium salt in a 1 wt. % acetic acid solution. Both solutions were left overnight at room temperature to ensure dissolution of the polymer. Upon dissolution, the two mixtures were combined and placed in a planetary centrifugal mixer (Thinky ARM-300, Thinky USA, Laguna Hills, CA) at 2000 rpm for 5 min before blending to homogenize the polymer solution. The CHA solution was blended twice for 5 min and cooled in an ice bath for 10 min in between blending steps.

CA and CHA scaffold solutions were cast into 96- or 384-well microplates via the automated scaffold dispenser. Scaffold solutions were loaded into a 60 mL syringe barrel (309654, BD, Franklin Lake, NJ). The syringes were fitted with precision, 20 gauge, Luer-locking dispensing tips (6699A4, McMaster-Carr, Los Angeles, CA). The syringe barrel was loaded into the scaffold dispensing device. The air pressure applied to the syringe barrel was adjusted using a pressure regulator; the exact applied pressure (60 kPa to 500 kPa) was uniquely adjusted for different percentages of scaffold (2, 4, and 8 wt. % polymer) to achieve 2 mm of scaffold in each well. After dispensing each well with scaffold solution, microplates were centrifuged (Srovall Legend XT, Thermo Scientific, Waltham, MA) at 1500 rpm for 1 min to degas air bubbles, placed in a freezer overnight at −20 °C for 24 h. Frozen scaffolds were lyophilized in a Labconco 6 freeze dryer for 1 to 3 days.

SEM images were acquired using an FEI Sirion XL830 Dual Beam FIB/SEM (FEI Company, Hillsboro, OR). Lyophilized specimens were cut in half, mounted on aluminum pin stubs (16111, Ted Pella Inc., Redding, CA) with carbon tape, and sputter coated with Au/Pd for 1 min at 18 mA before imaging. Images were taken with a 5 kV accelerating voltage, a spot size of 2, and 500× magnification.

The areas of the pores composing each scaffold were measured from representative SEM images using ImageJ software. Freehand selections were manually drawn around the perimeters of pores, and at least 60 pores per scaffold were analyzed to determine a median pore area for each scaffold. The distribution of pore areas within a scaffold represented as mean plus or minus the standard deviation.

The rheological properties of 4 wt. % CA and CHA polymer solutions were measured with a stress-controlled rheometer (MCR 301, Anton Paar, Germany). A parallel-plate geometry was used with a plate diameter of 25 mm. Viscosity was measured at a constant shear rate of 1 s-1 with a zero-stop gap of 1 mm at a constant temperature of 25 °C. Measurements were taken in 10 s intervals for 200 s.

U-118 MG human glioblastoma cells were purchased from the American Type Culture Collection (ATCC, Manassas, VA). Cells were maintained according to the supplier's protocol in fully supplemented DMEM with 10% FBS and 1% Pen Strep in a humidified incubator with 5% CO₂ at 37 °C.

U-118 MG cells (10,000 cells/well) were cultured in 4 wt. % CA scaffolds cast in 384-well microplates. Cells were treated with TMZ three days after cell seeding on scaffolds. Drug concentrations used for the trials were 156, 312, 625, 1250, 2500, and 5000 μM with n=12 wells per condition. Cell viability was investigated 3 days post-treatment with the alamarBlue assay. Briefly, 50 μL of alamarBlue solution (10% alamarBlue reagent in fully supplemented DMEM) was added to each well. Samples were incubated at 37 °C for 2 h, then, the alamarBlue solution was transferred to an opaque, black 96-well plate for fluorescent signal measurements using a SpectraMax M5 microplate reader (Molecular Devices, Union City, CA) at an excitation wavelength of 560 nm and a fluorescence emission read at 590 nm. Cell viability was reported as a percent of viable cells relative to control cells treated with DPBS. The drug response profile was estimated using nonlinear least square estimation of the three parameter Hill equation using the Imfit package in Python.

FIG. 10 is a graph of a drug-screening proof of concept using example scaffolds, in accordance with the present technology. On the vertical axis is the pore are in μm². On the horizontal axis is the wt. % of the scaffolds. Shown in FIG. 10 is 2 wt. %, 4 wt. %, and 8 wt. % scaffolds. As shown in FIG. 10, both 96- and 384-well plate scaffolds were tested. Further CA and CHA scaffolds were prepared and tested. Dose-dependent cytotoxicity of TMZ on U-118 MG glioma cells cultured in 4 wt. % CA scaffolds dispensed in a 384-well microplate. Cell viability was evaluated using the alamarBlue assay 3 days after treatment with TMZ. Data points (red circles) represent measured values whereas the dashed black line represents a best fit of the data to the Hill equation.

As shown in this example, an efficient and cost-effective scaffold dispensing system capable of quickly distributing high-viscosity solutions into miniature microplate formats with a high degree of uniformity in the resulting volume and 3D structure of the dispensed scaffolds from well to well was developed. This device may be extended for use with viscous materials other than those intended to serve as porous scaffolds for 3D cell culture such as foods and glues, and this platform may also be used with larger well plate formats such as a 24-well plate. Precise control over the magnitude and dwell time of the air pressure applied to a scaffold solution held within a syringe using software commands facilitates reliable and rapid dispensing. Dispensing time was decreased to 4 min in 96-well microplates and 15 min for 384-well plates, compared to 25 min and 90 min required for hand casting hydrogels in 96-well and 384-well microplates, respectively. Expedited and automated scaffold dispensing may open new avenues for high-throughput assays that use 3D cell culture techniques in lieu of 2D platforms. A proof-of-concept experiment was performed in this study where glioma cells cultured in dispensed scaffolds residing in a 384-well microplate were exposed to the chemotherapeutic TMZ, and these microplates were used to rapidly assay cell death using a fluorescence-based evaluation method. The 3D cell culture technique disclosed herein will help advance drug discovery by locating more efficacious and safer therapeutics, and that the high-throughput platform will accelerate clinical translation of potential drug candidates.

### Example #2: 3D Porous Scaffold-Based High-Throughput Platform for Cancer Drug Screening

Natural polymer-based porous scaffolds have been investigated to serve as three-dimensional (3D) tumor models for drug screening owing to their structural properties with better resemblance to human tumor microenvironments than two-dimensional (2D) cell cultures. In this example, a 3D chitosan-hyaluronic acid (CHA) composite porous scaffold with tunable pore size (60, 120 and 180 μm) was produced by freeze-drying and fabricated into a 96-array platform for high-throughput screening (HTS) of cancer therapeutics. A self-designed rapid dispensing system, such as the automated scaffold dispenser disclosed herein, was used to handle the highly viscous CHA polymer mixture and achieved fast and cost-effective large-batch production of the 3D HTS platform. In addition, the adjustable pore size of the scaffold can accommodate cancer cells from different sources to better mimic the in vivo malignancy. Three human glioblastoma multiforme (GBM) cell lines were tested on the scaffolds to reveal the influence of pore size on cell growth kinetics, tumor spheroid morphology, gene expression and dose-dependent drug response. The results showed that three GBM cell lines showed different trends of drug resistance on CHA scaffolds of varying pore size, which reflects the intertumoral heterogeneity across patients in clinical practice. The results also demonstrated the benefits of having a tunable 3D porous scaffold for adapting the heterogeneous tumor to generate the optimal HTS outcomes. It was also found that CHA scaffolds can produce uniform cellular response (CV<0.15) and wide drug screening window (Z′>0.5) on par with commercialized tissue culture plates, and therefore can serve as a qualified HTS platform. This CHA scaffold-based HTS platform may provide an improved alternative to traditional 2D cell-based HTS for future cancer study and novel drug discovery.

In this study, a self-designed rapid dispensing system (such as the automated scaffold dispenser described herein) was applied to manage the viscous polymer solution of chitosan and HA and establish a fast and cost-efficient approach to manufacture the HTS platform. The CHA scaffolds were prepared with a freeze-drying technique which is suitable for processing heat-sensitive natural polymers and capable of controlling the porous structure of the scaffold by adjusting freeze-drying parameters. Recent studies showed that changing the scaffold pore size alters the 3D microenvironment, affecting cell proliferation, migration, and differentiation. CHA scaffolds of different pore sizes were developed and explored to determine whether the tunable CHA scaffold can regulate the drug resistance of different cancer cell lines. The growth kinetics, gene expressions and does-dependent drug response of three GBM cell lines grown in the scaffold were assessed to reveal the effect of scaffold pore size on the drug resistance. The coefficient of variance and Z′ factor were also analyzed to verify the quality of the scaffold as a HTS platform. Disclosed herein is a novel 3D scaffold based HTS platform with great tunability to improve drug development efficiency and facilitate future cancer treatment.

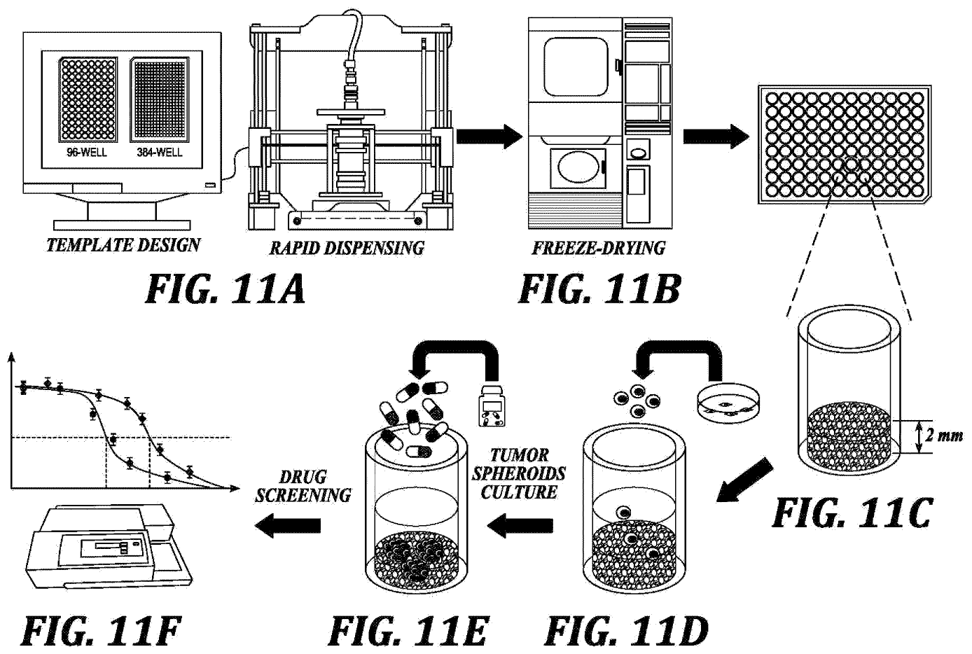

FIGS. 11A-11F are process blocks illustrating a method of making example scaffolds, in accordance with the present technology. The HTS platform was manufactured by computer-aided rapid dispensing of CHA polymer solution (FIG. 11A) and followed by freeze-drying to generate a 3D porous structure as shown in FIG. 11B. The design and setup of the dispenser is as described herein, where fully automated and well-controlled dispensing of viscous polymer solution into 96- and 384-well plates can be achieved. The CHA scaffolds were uniformly cast as 2 mm discs at the bottom of 96-well plates. FIG. 11C shows a single well of a well plate, having about 2 mm of viscous material (or scaffold material). In FIG. 11D, tumor spheroids are added to each well of the well plate. In FIG. 11E, the tumor spheroids have been cultured, and a drug is added. In FIG. 11F, drug screening occurs and is analyzed.

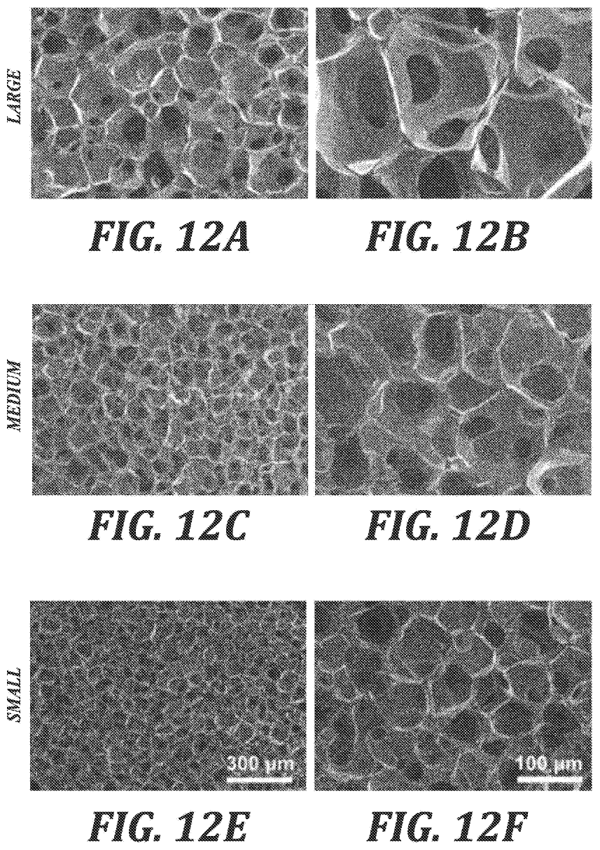

The rapid solution dispensing process takes less than one minute per plate and can be readily scaled up. The cost for both chitosan and HA is much lower than the cost of the materials for commercialized 3D cell culture matrices, such as Matrigel® or Geltrex™. Therefore, it holds great promise for economic 3D HTS platform manufacture. The different scaffold pore sizes were achieved by altering the freezing-annealing time, while keeping the same polymer concentration. Different freezing history and annealing time can create distinct ice crystal structures in chitosan-HA mixture, leading to varied scaffold pore size after dehydration. The density of all three scaffolds were measured, and they showed similar density of approximately 0.075 g/cm³. We then examined the scaffolds with a scanning electron microscope (SEM) to reveal their different microscopic structures. As seen in FIG. 2a, three CHA scaffolds showed very distinct pore sizes while all comprised highly interconnected pores and uniform pore shapes. The scaffolds with large pore size (CHA-L), medium pore size (CHA-M) and small pore size (CHA-S) have an average pore diameter of ~186.58 μm, ~119.77 μm and 63.57 μm, respectively, as shown in FIG. 12B. These results demonstrated the excellent tunability of the CHA scaffold structure made by the freeze-drying method.

FIGS. 12A-12F show the scaffold microstructure of dispensed scaffolds, in accordance with the present technology. From top to bottom, SEM images of scaffolds with large pore (CHA-L—FIGS. 12A-12B), medium pore (CHA-M—FIGS. 12C-12D) and small pore (CHA-S—12E-12F) under 100× (right column—FIGS. 12A, 12C, and 12E) and 300× (left column—FIGS. 12B, 12D, and 12F) magnification.

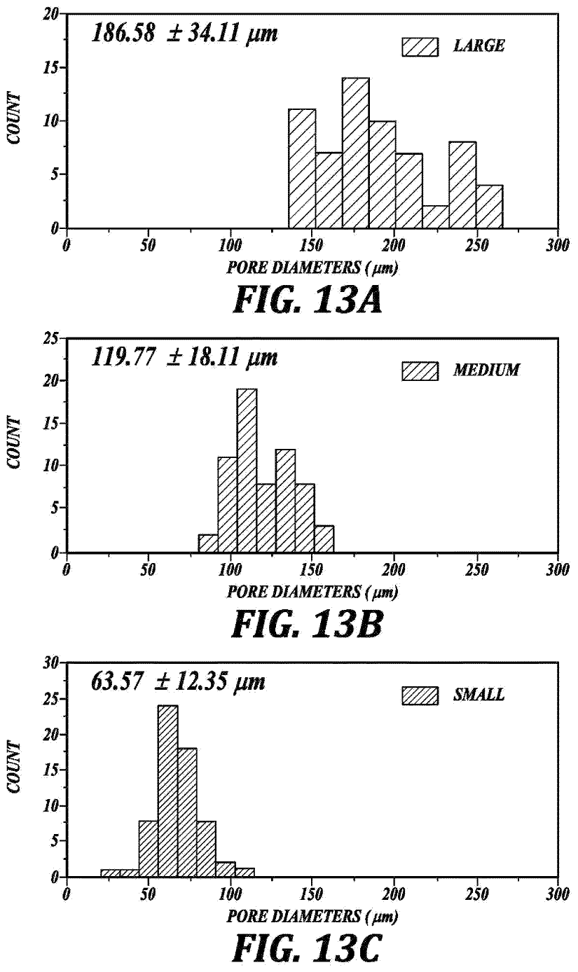

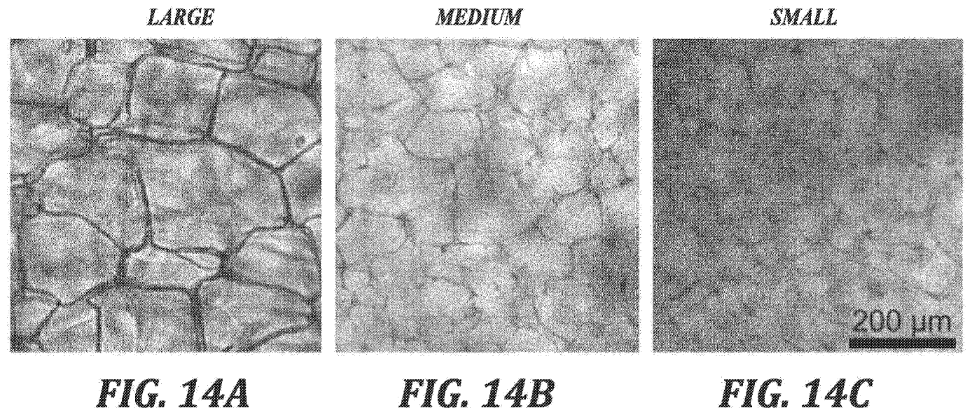

FIGS. 13A-13C are graphs of pore size distributions of the dispensed scaffolds of FIGS. 12A-12F, in accordance with the present technology. On the vertical axis is the number of pores of each scaffold. On the horizontal axis is pore diameter in μm. FIG. 13A shows large pore scaffolds, FIG. 13B shows medium pore scaffolds, and FIG. 13C shows small pore scaffolds. At least 50 pores were measured for each scaffold. The structure stability of 3D porous scaffolds during cell culture is useful for appropriate cell attachment, proliferation, and consistent cellular responses. It is imperative for scaffolds to maintain an intact pore structure, stable pore size and good interconnectivity under physiological conditions.

FIGS. 14A-14C are porous structures of example rehydrated dispensed scaffolds, in accordance with the present technology. Specifically, FIGS. 14A-14C are optical images of CHA scaffolds with large, medium, and small pores, after hydrated by PBS for 7 days.

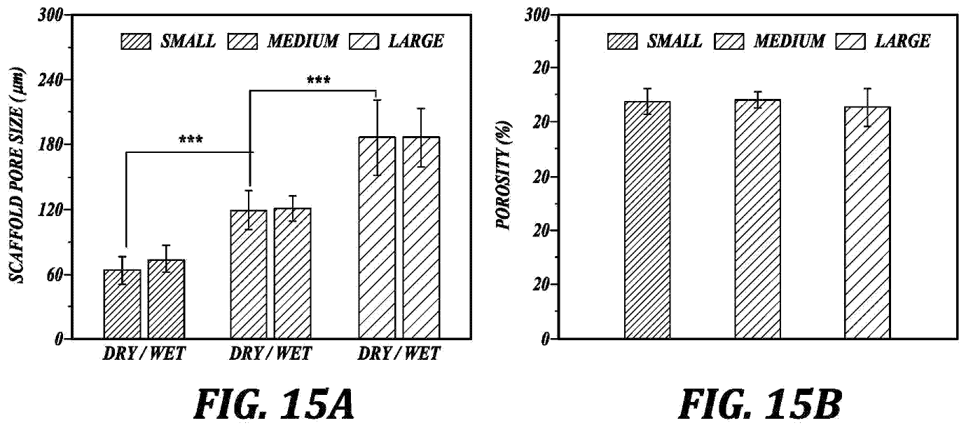

FIGS. 15A-15B are graphs of porosity of the example dispensed scaffolds of FIGS. 14A-14C, in accordance with the present technology. FIG. 15A is a graph showing the scaffold pore size in dry and wet conditions. n>10. On the vertical axis is the scaffold pore size in μm. On the horizontal axis are dry and wet scaffolds, having various pore sizes (small, medium, and large). FIG. 15B is a graph showing the porosity of scaffolds of three different pore sizes measured by liquid displacement using isopropanol. n=5. ***p<0.001. On the vertical axis is the porosity in percentage. Here, the structure stability of the scaffolds was first examined by comparing the hydrated scaffolds with the dry scaffolds. CHA scaffolds were hydrated and incubated in PBS at 37 °C for 7 days before imaged optically, as shown in FIGS. 14A-14C. As shown, the CHA scaffolds of three different sizes (small, medium and large) incubated under physiological conditions remained uniform in pore structure one week after incubation in PBS. The hydrated scaffolds showed virtually no changes in pore size as compared to their dry counterparts, suggesting that the scaffolds bear excellent structure stability. FIG. 15B shows a comparison of scaffold porosity between the scaffolds of three pore sizes. All three scaffolds showed high open porosity (~90%) and no significant difference in porosity was found. The high scaffold open porosity is essential to accommodate cell proliferation. An open and interconnected pores inside CHA scaffolds allows for proper exchange and diffusion of nutrients, oxygen, and drug molecules, which support a healthy cell status and also accurate cellular response to drug treatment.

In addition to the stability, the stiffness of scaffolds has a strong influence on cell adhesion, morphology as well as cell signaling pathways. The mechanical environments sensed by cancer cells largely influence the cancer cell spreading and metastasis. Therefore, the mechanical properties of CHA scaffolds with different pore sizes were further evaluated. The compressive strength and modulus of different CHA scaffolds was then measured.

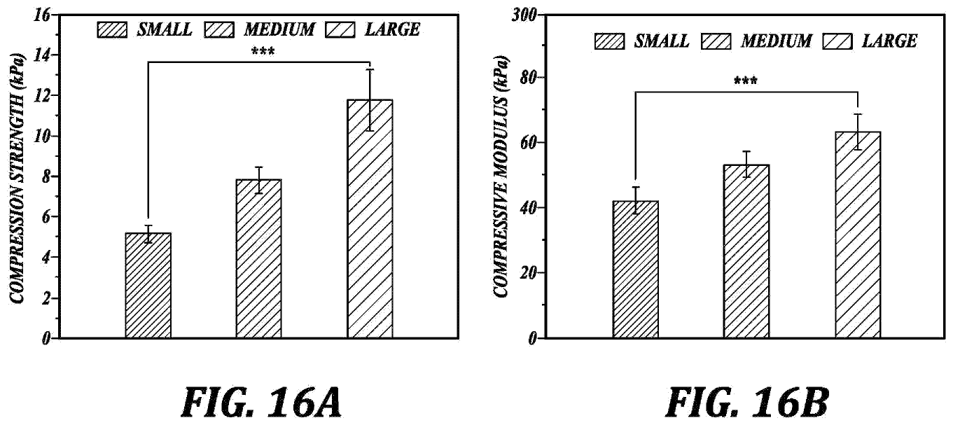

FIGS. 16A-16B are graphs of the mechanical properties of the example dispensed scaffolds of FIGS. 14A-14C, in accordance with the present technology. In FIG. 16A, the vertical axis is compressive strength in kPA. In FIG. 16B, on the vertical axis is compressive modulus in kPA. Small, medium, and large pore scaffolds are shown in both FIGS. 16A and 16B. As shown in FIGS. 16A-16B, CHA scaffolds of the largest pore size have the highest compressive strength (11.74±1.52 kPa) and modulus (63.30±5.44 kPa), while scaffolds with smallest pore size have the lowest strength (5.19±0.42) and modulus (42.17±4.04 kPa). This agrees with results from previous studies in that an increase in pore size is accompanied by an increase in stiffness for bulk scaffolds. Nevertheless, the surface Young's modulus has been reported to determine how a single cell senses its surrounding matrix. It was also reported that local surface mechanical properties of the pore walls of scaffolds are independent of varying pore size. Therefore, the surface Young's modulus of the scaffolds using atomic force microscopy (AFM) was further observed, and nearly identical surface Young's modulus were measured for three scaffolds. Thus, it was suggested that cancer cells incubated in CHA scaffolds with different pore sizes experience a similar micro-mechanical environment. Furthermore, the surface charge of scaffolds may also alter cell adhesion, migration, and proliferation behaviors through regulating the surface protein absorption. The zeta potentials of the three scaffolds were measured, and they showed no significant difference under physiological pH, owing to that all three scaffolds were prepared by the same materials and concentration. These results provided clear evidence that the micro-mechanical environment and surface properties of CHA scaffolds are independent from changes in pore size, suggesting which would be the only factor that may alter the tumor microenvironment and regulate the cellular behaviors in the 3D CHA scaffolds.

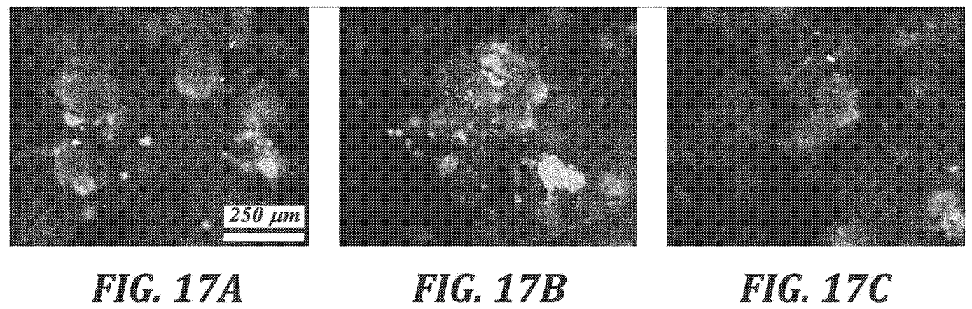

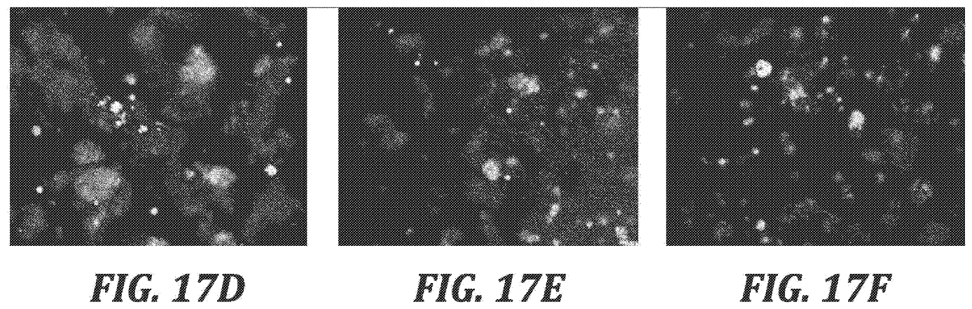

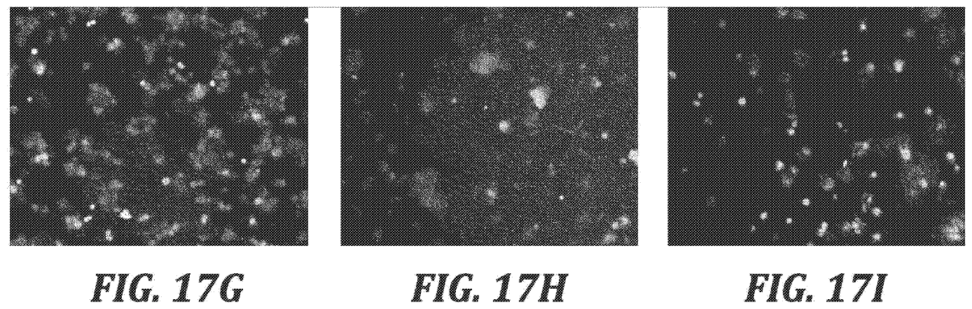

FIGS. 17A-17I are fluorescent images of live and dead cells of three example scaffolds having different pore sizes (small, medium, and large), in accordance with the present technology. FIGS. 17A-17I show fluorescence images of live/dead cells of U87, U118 and GBM6 grown in CHA scaffolds with different pore sizes for 7 days. **p<0.01, ***p<0.001.

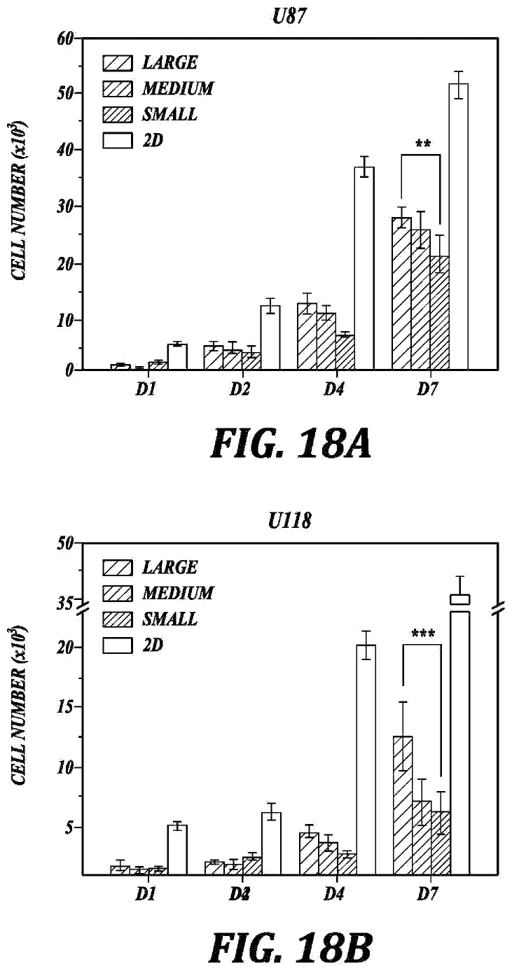

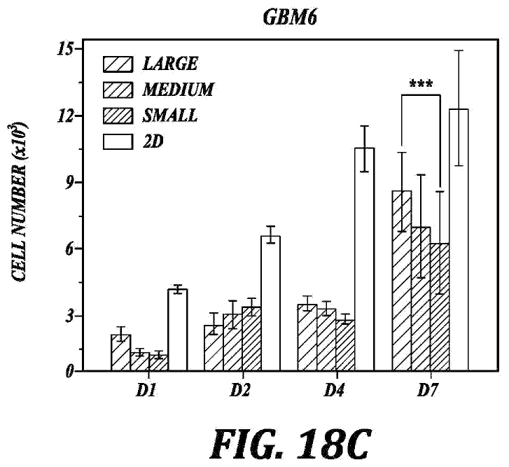

FIGS. 18A-18C are graphs of cell proliferation profiles on two dimensional (2D) microplates and in scaffolds of different pore sizes as shown in FIGS. 17A-17I, in accordance with the present technology. FIG. 18A is a graph of cell proliferation for U87. FIG. 18B is a graph of cell proliferation for U118. FIG. 18C is a graph of GBM6 cell proliferation. Each profile occurred on a 2D micro-plates and in CHA scaffolds of small, medium, and large pore sizes, after 7 days of culture. n=5. On the vertical axis of each FIG. 18A-18C is cell number in 103. On the horizontal axis of each graph is time in days (day 1—D1, day 2—D2, day 4—D4, and day 7—D7).

Besides cell proliferation, it is worthwhile to investigate the influence of the CHA scaffolds with varying pore size on the tumor drug resistance as a drug screening platform. Hence, the drug resistance-associated gene expressions of three cell lines cultured in CHA scaffolds were further examined and compared with 2D counterparts. Cells were cultured in CHA scaffolds for 7 days and then collected. Three tumor malignancy-associated genes, the chemoresistance markers ABCG2 and MGMT, and the tumor invasive marker CD44 were analyzed by RT-qPCR. The ABCG2 is a member of the ABC-drug transporter family, which can actively efflux drugs from cells, serving to protect them from cytotoxic agents through ATP hydrolysis. The MGMT encodes the DNA-repair protein O6-alkylguanine (O6-AG) DNA alkyltransferase (AGT), which repairs the alkylating lesion caused by chemo drugs such as TMZ. The CD44 represents a cell surface receptor for hyaluronate, which modulates the tumor invasion and metastasis.

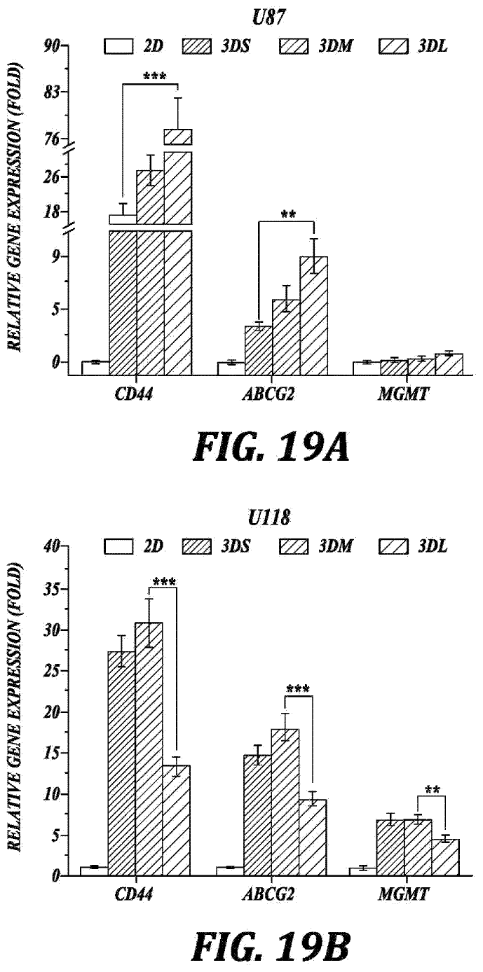

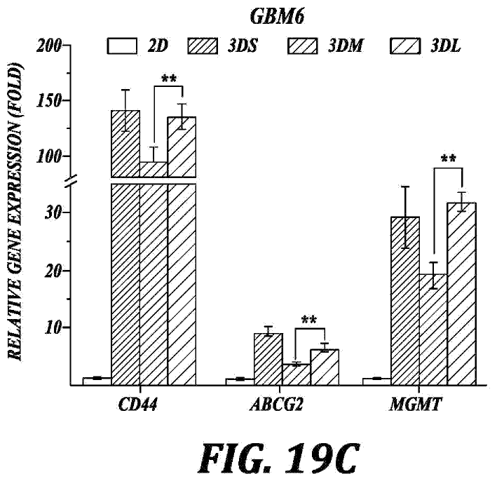

FIGS. 19A-19C are graphs showing gene expression of (GBM) cells on different substrates, in accordance with the present technology. The expression of drug resistance-associated genes of U87 cells (FIG. 19A), U118 cells (FIG. 19B), and GBM6 cells (FIG. 19C) on 3D CHA scaffolds of different pore sizes relative to 2D culture, respectively. n=3. **p<0.01, ***p<0.001. On the vertical axis of each graph is the relative gene expression in folds. On the horizontal axis are chemoresistance markers CD44, ABCG2, and MGMT. 2D, 3D scaffolds with small pores, medium pores, and large pores are shown in FIGS. 19A-19C.

Interestingly, the gene expression of three GBM cell lines showed different trends in CHA scaffolds of varying pore sizes. FIGS. 19A-19C shows the expression of the above-mentioned genes for the three GBM cell lines cultured on different substrates relative to 2D controls. For U87 (FIG. 19A), the examined genes exhibited highest expression in CHA-L scaffolds, followed by CHA-M in the middle and CHA-S scaffolds in the least, indicating that the larger pore size is supporting U87 cells to develop more drug resistant and aggressive phenotypes. For U118 (FIG. 19B), on the contrary, it was observed that the lowest gene expression in the CHA-L scaffolds, and the CHA-M had the highest expression level. For GBM6 (FIG. 19C), the gene expression was higher in both CHA-L and CHA-S scaffolds, while the gene expression in CHA-M remained the lowest. The genetic heterogeneity of tumors has been long confounding clinical diagnosis and posing great challenges to the development of effective therapy for cancer treatment. By changing the CHA scaffold pore size, the tumor microenvironment was altered, which is one of the major sources of tumor heterogeneity. Comparing the three GBM cell lines grown in different CHA scaffolds, the gene expression was regulated differently in terms of the invasiveness, malignancy, and drug resistance genotypes, which presents a good example of the intertumoral heterogeneity. Therefore, by fine tuning of the scaffold pore size, it was possible to better recapitulate the most malignant tumor in the in vivo environment and improve the reliability of the HTS outcomes in vitro.

The cellular response of an in vitro tumor model to chemotherapy drugs is affected by a complex synergic effect of different tumor genotypes, hierarchies, cell-cell communications, and cell-matrix interactions. The wide differences in the biophysical characteristics of different tumor cells present a significant challenge to determining the optimal HTS platform that can provide the best drug screening accuracy and efficacy. As shown in the previous sections, the scaffold pore size is a factor regulating an in vitro 3D tumor model, affecting the tumor proliferation and altering the gene expressions. Thus, a tunable scaffold pore size is essential to accommodate different cell lines for optimal drug screening outcomes.

Here, the drug resistance of the three GBM cell lines was further analyzed based on the cell viability under a dose-dependent drug response test. Three cell lines were cultured in three CHA scaffolds respectively and tested using a standard anti-cancer drug for GBM therapy, temozolomide (TMZ). The GBM cells were first allowed to grow in scaffolds for 7 days to adapt to different pore sizes and form tumor spheroids. The tumor spheroids were then exposed to TMZ for 72 hours, and the dose-dependent cell viability was then evaluated as the percentage metabolic activity compared to non-treated controls on the same substrate respectively. The half maximal effective concentration (EC50) was calculated to quantify the drug resistance.

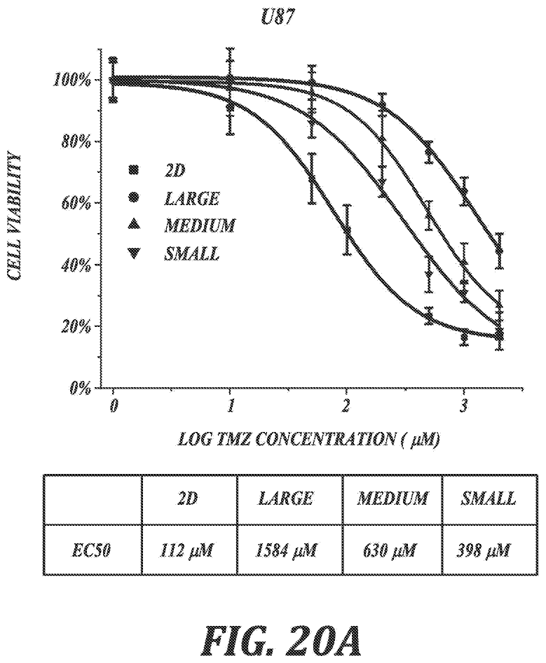

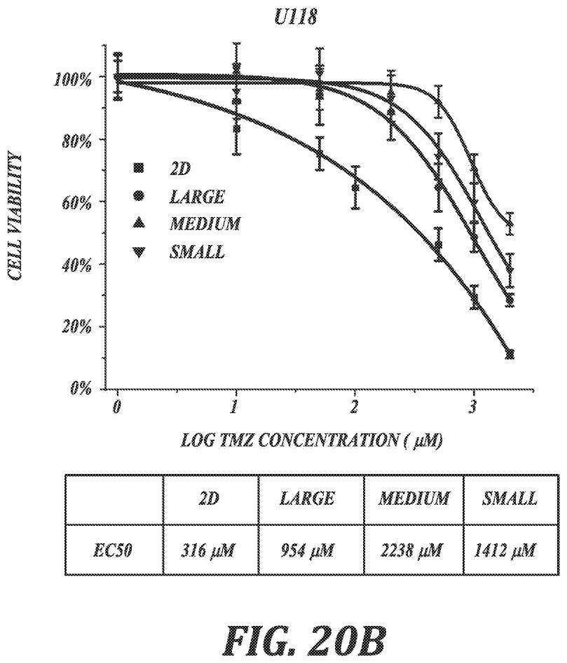

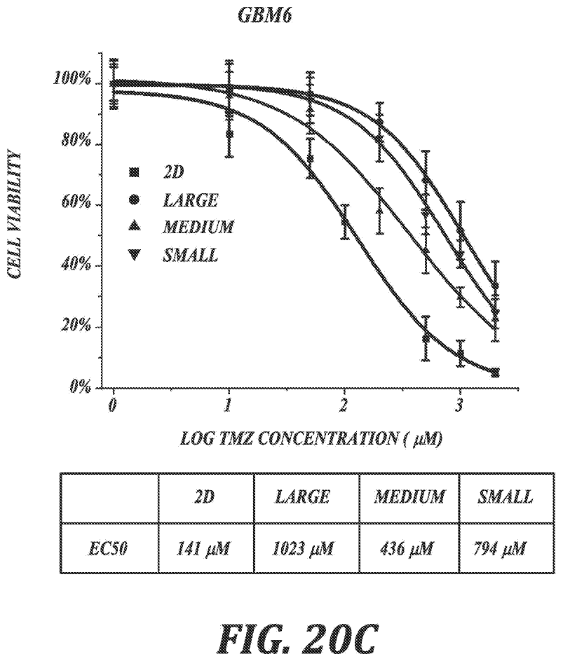

FIGS. 20A-20C are graphs of drug responses of GBM cells on different substrates, in accordance with the present technology. FIGS. 20A-2C show dose-dependent response of U87 (FIG. 20A), U118 (FIG. 20B) and GBM6 (FIG. 20C) grown on different substrates after 7 days and treated with TMZ for 72 hours. Cell viability was determined using AlamarBlue assay and normalized to untreated groups. n=5. Also shown is the EC50 for TMZ treatment of U87 (FIG. 20A), U118 (FIG. 20B) and GBM6 (FIG. 20C) on 2D micro-plates and CHA scaffolds of different pore sizes. Similar to the gene expression results, three cell lines had distinct cellular responses to TMZ in scaffolds with different pore sizes. As shown in FIG. 20A, the U87 showed the highest survival rate in CHA-L scaffolds, followed by CHA-M and CHA-S. On the contrary, U118 (FIG. 20B) showed lowest survival in CHA-L, with CHA-M being in the highest and CHA-S being the middle. For GBM6, the cells in CHA-L and CHA-S scaffolds had higher survival rates than those in CHA-M scaffolds (FIG. 20C). The three cell lines all showed higher survival rates in 3D CHA scaffolds than in their 2D counterparts regardless of pore size, providing evidence that the cells rapidly obtained different levels of drug resistance to TMZ in 3D cultures. To be noted, the cell survival rates of three GBM cell lines exhibited a good match with the drug resistance associated gene expression. These data further demonstrated that drug resistance of an in vitro tumor model can be altered by tuning the pore size. Thus, the use of CHA scaffolds as a HTS platform allows us to create an in vitro tumor model with the highest level of malignancy, presenting unique opportunities to enhance drug screening efficacy.

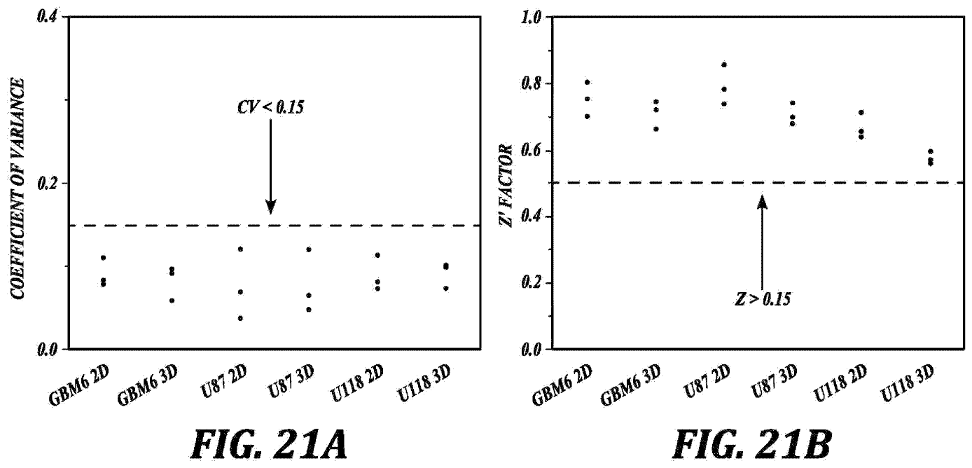

The criteria for assessing the quality of a HTS platform is usually referring to two parameters, the cellular response uniformity, and the drug screening window on 96- or 384-well plate-based drug screening. The uniformity is characterized by coefficient of variance (CV), which is determined as the ratio of the standard deviation to the mean value of the cellular response across plates. The drug screening window is quantified by a coefficient termed “Z′ factor”, which reflects the data variation level and the cellular response signal dynamic range of control groups. Z′ factor is a characteristic parameter to assess the quality of an HTS platform based on properly selected positive and negative controls, without intervention of test compounds. Here, we investigated the quality of our CHA scaffold based HTS platform to compare with commercialized 2D tissue culture plates following a previous reported method with minor modification. CHA scaffolds with medium pore size were picked as a sample platform, 0.1% DMSO and 2 mM TMZ were used as negative and positive control for all three cell lines on both 3D and 2D cultures. As shown in FIG. 8a, all three cell lines cultured in CHA scaffolds showed relatively low CV and were within the acceptable degree of variance for in vitro cell-based assay (<0.15). In addition, the performance of the CHA scaffold platform was on par with commercialized 2D micro-plates. A Z′ factor above 0.5 is deemed as an excellent screening window between positive and negative controls. As shown in FIG. 21B, all three cell lines on 3D culture conditions showed a high Z′ factor value above 0.5. Together with the CV data, this proves that the CHA scaffold serves as a high quality HTS platform comparable with commercialized 2D tissue culture plates. All chemicals were purchased from Merck (USA) unless otherwise specified.

8% CHA scaffolds were prepared similarly to a previously described method [9]. 8% w/w chitosan (Medical grade, Matexcel, USA) and 1% w/w hyaluronic acid (hyaluronic acid sodium salt, from Streptococcus equi) were fully dissolved in 1% w/w acetic acid aqueous solution, respectively. Two solutions were then combined and mixed using a Thinky mixer (ARM-300, Thinky, USA) at 2000 rpm for three minutes, and further mixed in a blender for ten minutes to ensure a homogeneous polymer mixture. The polymer mixture was then centrifuged at 2000 rpm for 30 min to remove air bubbles and cast into 96-well tissue culture plates using a self-designed computer-aided automated scaffold dispenser. The dispensing pressure and dispensing time were controlled to achieve a dispensing of 65 μl/well in order to reach a thickness around 2 mm. The total dispensing time for each 96-well tissue culture plate is 40-50 seconds. To produce scaffolds with large pore size, the plates were first frozen at −20 °C for 24 h, then thawed at ambient temperature for 2 h. The plates were then carefully transferred into a SP VirTis Genesis Pilot Lyophilizer (SP Scientific, USA). The plates were frozen in the lyophilizer using the following settings: 0 °C for 60 min, ramp to −5 °C in 40 min and stay for 20 min, ramp to −20 °C in 15 min and stay for 30 min, ramp to −2 °C in 18 min and stay for 24 h. To produce scaffolds with medium pore size, the plates were directly transferred into the lyophilizer and using the following freezing setting: 0 °C for 60 min, ramp to −5 °C in 40 min and stay for 20 min, ramp to −20 °C in 15 min and stay for 30 min, ramp to −2 °C in 18 min and stay for 4 h. To produce scaffolds with small pore size, the plates were directly transferred into the lyophilizer and using the following freezing setting: 0 °C for 60 min, ramp to −5 °C in 40 min and stay for 20 min, ramp to −70 °C in 65 min and stay for 30 min, ramp to −2 °C in 68 min and stay for 1 h. After frozen, the plates were lyophilized under 100 mTorr at −1 °C until scaffolds were fully dehydrated. The scaffolds were neutralized in 7% v/v ammonium hydroxide/methanol solution for 30 min under vacuum, rinsed intensively with DI water and soaked in PBS for 24 h to remove residual base. The scaffolds were then sterilized using 70% ethanol for 24 h and then washed with sterilized PBS three times and incubated in 37 °C for another 24 h prior to cell seeding.

Scaffold imaging and pore size characterization: Scaffolds were sectioned into 400 μm thin slices using a Compresstome VF-300 Vibrating Micromtome (Precisionary, USA). Samples for SEM were sputter-coated with gold/platinum before imaging. SEM images were captured under 100× and 300× magnification using a JSM-6010 Plus scanning electron microscope (JEOL, Japan). Samples for optical imaging were hydrated with PBS for 7 days before imaging. Optical images were captured using a MU-1000 optical microscope (Amscope, USA). The scaffold pore size was characterized by measuring the individual pore diameter using ImageJ software. At least 50 pores were measured for each scaffold.

The scaffold open porosity was measured by liquid displacement method as previously described. Briefly, dry scaffold volume (Vi) and weight (Wi) were first recorded. The scaffold was then immersed in isopropanol (with known density ρi) under vacuum until the scaffold stopped bubbling and sank to the bottom. The impregnated scaffold was carefully wiped off excessive isopropanol and weighed again to get the final weight (Wf). The change in the volume of the impregnated scaffold was deemed to be negligible as isopropanol is nonsolvent. The open porosity was defined as the ratio of volume of solvent within the scaffold pores to the volume of the dry scaffold as shown in Equation (1).

$$Open Porosity = \frac{( {W}_{f} - {W}_{i} ) / {ρ}_{i}}{{V}_{i}} \times 100 \% \qquad Equation ( 1 )$$

The scaffolds for compressive strength and modulus measurement were prepared in 24-well plates with the same freezing setting as described above. Scaffolds were hydrated with PBS and trimmed into cylindrical shapes with 10 mm in height and 14 mm in diameter. The compression test was conducted at room temperature using a Shimadzu universal tester (AGS-X Series, Shimadzu, Japan) with a rate of 0.4 mm/min until at least 50% strain was obtained. The compressive strength was determined as the compressive stress at the yield point. The compressive modulus was determined as the slope of the linear region of the stress-strain curve. The scaffolds for surface Young's modulus measurement were sectioned into 400 μm thin slices and hydrated with PBS prior to measurement. The surface Young's moduli of scaffolds were obtained by conducting the nanomechanical measurement using a EasyScan atomic force microscope (Nanosurf AG, Switzerland). The measurement was performed under aqueous environment using a ContAl-G (BudgetSensor, Bulgaria) silicon nitride tip in contacting mode. Each sample was scanned in an 8×8 array at three different 1 nm2 square areas. The surface Young's modulus was calculated based on force-displacement curves.

The zeta potential of scaffolds was analyzed using a SurPASS 3 electrokinetic analyzer (Anton Paar, USA). Briefly, scaffolds were cast and freeze-dried into 1 mm thick sheets in 60 mm Petri dishes. The scaffolds were then neutralized, rinsed and trimmed to fit the size of the sample tube of the electrokinetic analyzer. The zeta potentials of scaffolds were measured in triplicate using 0.01 M KCl as buffer solution at pH=7.4 to reflect the surface charge in physiological conditions.

The human glioblastoma cell lines U-87 MG and U-118 MG were purchased from American Type Culture Collection (ATCC, USA). The human glioblastoma cell GBM6 was previously established in our laboratory. Cells were seeded on 2D 96-well plates and PBS-damped CHA scaffolds with different pore sizes in 96-well plate at 5000 cells per well and cultured in 100 μl fully supplemented medium (Dulbecco's Modified Eagle Medium with 10% Fetal bovine serum and 1% antibiotic-antimycotic). The cell metabolic activities were monitored on day 1, 2, 4, 7 after seeding using the AlamarBlue metabolic assay and following the manufacturer's protocol (Life Technologies, USA) [54-56]. Briefly, AlamarBlue stocking solution was diluted ten times using fully supplemented medium and then added to each well (150 μl) and incubated at 37 °C for 2 h. Next, 100 μl of the AlamarBlue solution was transferred from the cell-culture plate to an opaque 96-well plate and the fluorescence intensity was measured using a VersaMax Microplate Reader (Molecular Devices, USA). The cell number was calculated based on previously generated standard curves.

The cell morphology in scaffolds with different pore sizes was studied by observing the shape of tumor spheroid using live/dead imaging. The scaffolds were sectioned into 400 μm slices for better transparency prior to cell seeding. Cells were seeded on CHA scaffolds in 96-well plate at 5000 cells per well and cultured for 7 days in fully supplemented medium. After day 7, the fully supplemented medium was replaced by fluorescence dyes (PBS containing 0.1% v/v Calcien AM and 0.1% v/v PI), and then incubated for 30 min before imaging. Next, the scaffolds were mounted to microscope slides. 10 μl of fluorescence dyes were added to prevent scaffolds from drying and covered with the coverslips immediately. Fluorescence images were captured using a Nikon TE300 (Nikon, Japan) inverted microscope.

Cells were seeded on 2D 96-well plates and scaffolds in 96-well plates at 5000 cells per well and cultured in fully supplemented medium for 7 days. After day 7, cells were collected from 2D plates and scaffolds using TriplE express. The RNA was extracted using Qiagen RNeasy kit (Qiagen, Germany) following the manufacturer's protocols. The reverse transcription was conducted using iScript cDNA synthesis kit (Bio-Rad, USA) following the manufacturer's protocols to produce cDNA. Thermocycling was performed in 20 μL solution system with 10 μl of SYBR Superrmix (Bio-Rad, USA), 2 μl of 10 nM primers, 7 μl of DNase-free water and 1 μl of 50 ng/μl cDNA. The qRT-PCR was conducted on a CFX96 Touch Real-Time PCR Detection System (Bio-Rad, USA). The thermocycle was set as 95 °C for 2 min, 40 cycles at 95 °C for 15 s, 58 °C for 30 s, and 72 °C for 30 s. Data were analyzed with the CFX Manager software (Bio-Rad, USA) with expression levels normalized to GAPDH. The primers (Integrated DNA Technologies, USA) were listed in Table 2.

TABLE 2
Primers used for PCR.

| Target | Forward (5′-3′) | Reverse (5′-3′) |
|---|---|---|
| GAPDH | ACCACAGTCCATGCC | TCCACCACCCTGTTG |
| | ATCAC | CTGTA |
| CD44 | CCAGAAGGAACAGTG | ACTGTCCTCTGGGCT |
| | GTTTGGC | TGGTGTT |
| ABCG2 | GTTCTCAGCAGCTCT | TCCTCCAGACACACC |
| | TCGGCTT | ACGGATA |
| MGMT | CCTGGCTGAATGCCT | GCAGCTTCCATAACA |
| | ATTTCCAC | CCTGTCTG |

Cells were seeded on 2D 96-well plates and CHA scaffolds in 96-well plates at 5000 cells per well and cultured in fully supplemented medium for 7 days. After day 7, the fully supplemented medium was replaced with TMZ solution with a gradient concentration of 0 (with 0.1% v/v DMSO), 10, 50, 100, 200, 500, 1000, 2000 μM and treated for 72 h. After 72 h, the cell metabolic activities were measured by the AlamarBlue assay as described previously. The relative cell viability was determined as the relative metabolic activities to the untreated control groups.

The screening window coefficient, Z′ factor, was calculated to evaluate the quality of CHA scaffolds as an HTS platform. The coefficient of variance, CV was also calculated to analyze the result consistency. CHA scaffolds with medium pore size were used for HTS validation. Briefly, cells were seeded on 2D 96-well plates and 8% CHA scaffolds in 96-well plates at 5000 cells per well and cultured in fully supplemented medium for 7 days. After day 7, cells were treated with 2000 μM TMZ solution as the positive control, and 0 μM TMZ (with 0.1% v/v DMSO) as the negative control for 72 h. After 72 h, the cell metabolic activities were measured by the AlamarBlue assay as described previously. The cell metabolic activities quantified by fluorescence intensities were used to calculate the Z′ factor and CV. CV and Z′ for each cell line were obtained from three independent micro-plates. The CV was calculated by dividing standard deviation of fluorescence intensity with average intensity. The Z′ was calculated using Equation (2).

FIGS. 21A-21B are graphs characterizing the quality of the scaffolds, in accordance with the present technology. FIG. 21A shows the coefficient of variance calculated for three GBM cell line viability in response to TMZ treatment on both 2D and CHA scaffolds with medium pore size. n=3. On the vertical axis is the coefficient of variance. On the horizontal axis is a series of cells in 2D and 3D scaffolds (GBM6, U87, U118). FIG. 21B shows the Z′ factor calculated for GBM cell viability in response to TMZ treatment on both 2D and CHA scaffolds with medium pore size. n=3. On the vertical axis is the Z′ factor. On the horizontal axis is a series of cells in 2D and 3D scaffolds (GBM6, U87, U118).

$${Z}^{′} = 1 - \frac{3 ( {σ}_{p} + {σ}_{n} )}{\vert {μ}_{p} - {μ}_{n} \vert} \qquad Equation ( 2 )$$

where σp and μp are defined as standard deviation and mean of the fluorescence intensity in positive controls. σn and μn are defined as standard deviation and mean of the fluorescence intensity in negative controls.

All data were presented as the mean±standard deviation (SD) of the mean. p<0.05 was set as statistical significance and was tested with Student's t-test (GraphPad Prism).

In this study, the self-designed rapid dispensing system (or automated scaffold dispenser as described herein) was used to achieve a fast and cost-effective large-batch production of 3D CHA porous scaffolds as a HTS platform. The 3D CHA porous scaffolds fabricated with adjustable pore size using a freeze-drying technique showed significant difference in pore dimension, and similar surface charge and micromechanical environment. Three GBM cell lines on the CHA scaffolds with different pore sizes and investigated the effect of pore size on the cell proliferation, morphology, and gene expression were studied. Significantly, a dose-dependent drug treatment using TMZ on three cell lines in CHA scaffolds with varying pore size was conducted, and discovered the results collectively capture the intertumoral heterogeneity of drug resistance. The heterogeneous nature of tumors is a major challenge in drug discovery and clinical trials. The tunable HTS platform holds the potential to improve drug screening outcomes for different cancer cell lines by allowing us to adjust the pore size to achieve the highest drug resistance. In addition, it has been demonstrated that the high quality of the 3D HTS platform by validating comparable CV and Z′ factor values of three GBM cell lines tested with TMZ on CHA scaffolds and commercial tissue culture plates. Therefore, the 3D CHA scaffold based HTS platform stands as a powerful tool for future cancer research and new anti-cancer therapeutic development.

### Embodiments

Embodiment 1: A viscous material dispenser, including an extruder comprising an inlet, a barrel, and a nozzle; a pump configured to fit inside the barrel; a tube connected to the inlet, wherein the tube branches into at least two arms; a first valve attached to a first arm of the tube, wherein the first valve opens and closes to a source of pressure; and a second valve attached to a second arm of the tube; wherein when the first valve is open and the second valve is closed, the pump is depressed by the source of pressurized air, causing a viscous material to flow out of the nozzle.

Embodiment 2: The viscous material dispenser of Embodiment 1, wherein when the first valve is closed and the second valve is open, the viscous material stops flowing out of the nozzle.

Embodiment 3: The viscous material dispenser of Embodiment 1 or 2, wherein the first valve and/or the second valve is selected from a pneumatic valve, a peristaltic valve, a piezoelectric valve, a hydraulic valve, an electromagnetic pump, or a progressive cavity valve.

Embodiment 4: The viscous material dispenser of any one of Embodiments 1-3, wherein the source of pressure is pressurized gas.

Embodiment 5: The viscous material dispenser of any one of Embodiments 1-3, wherein the source of pressure is pressurized liquid.

Embodiment 6: The viscous material dispenser of any of Embodiments 1-5, wherein the pump is a plunger.

Embodiment 7: The viscous material dispenser of any of Embodiments 1-5, wherein the pump is a progressive cavity pump.

Embodiment 8: The viscous material dispenser of any one of Embodiments 1-7, wherein the viscous material dispenser further comprises a stepper-motor powered moveable stage configured to move a substrate horizontally and vertically in precise step sizes so that one of the wells of the substrate is always directly beneath the nozzle of the extruder when the stepper-motor powered moveable stage is stationary.

Embodiment 9: The viscous material dispenser of any one of Embodiments 1-8, wherein the viscous material dispenser is multiplexed.

Embodiment 10: The viscous material dispenser of any one of Embodiments 1-9, wherein the second valve opens and closes to the atmosphere.

Embodiment 11: The viscous material dispenser of any one of Embodiments 1-10, wherein the viscous material dispenser further comprises a heater configured to heat the viscous material to a constant temperature to decrease the viscous material's viscosity.

Embodiment 12: The viscous material dispenser of Embodiment 11, wherein the heater comprises: a heating pad; and a metal tube surrounding the barrel and mounted to the heating pad.

Embodiment 13: The viscous material dispenser of Embodiment 12, wherein the metal tube is an aluminum tube.

Embodiment 14: The viscous material dispenser of any of Embodiments 11-13, wherein the heater further comprises a heating jacket surrounding the metal tube.

Embodiment 15: The viscous material dispenser of any one of Embodiments 11-14, wherein the heater heats the viscous material to a temperature between 20 °C and 80 °C.

Embodiment 16: The viscous material dispenser of any one of Embodiments 1-15, wherein the first valve and the second valve are a first solenoid and a second solenoid, respectively.

Embodiment 17: The viscous material dispenser of any one of Embodiments 1-16, wherein the extruder is a syringe.

Embodiment 18: A method of dispensing a viscous material using a viscous material dispenser according to any of the preceding embodiments, the method comprising: positioning a substrate underneath the nozzle; filling the barrel of the extruder with a viscous material; and dispensing the viscous material from the nozzle of the extruder and onto the substrate by opening the first valve and closing the second valve.

Embodiment 19: The method of Embodiment 18, the method further comprising: stopping the dispensing of the viscous material by closing the first valve and opening the second valve.

Embodiment 20: The method of Embodiment 18 or Embodiment 19, the method further comprising centrifuging the substrate.

Embodiment 21: The method of any one of Embodiments 18-20, the method further comprising lyophilizing the substrate to provide a cell scaffold.

Embodiment 22: The method of any one of Embodiments 18-21, wherein the substrate is a microplate.

Embodiment 23: The method of Embodiment 22, wherein the microplate is a 96-well microplate.

Embodiment 24: The method of Embodiment 22, wherein the microplate is a 384-well microplate.

Embodiment 25: The method of any one of Embodiments 18-24, the method further comprising: actuating a stepper-motor powered moveable stage horizontally and vertically in precise step sizes so that one of the wells of the substrate is always directly beneath the nozzle of the extruder when the stepper-motor powered moveable stage is stationary.

Embodiment 26: The method of any one of Embodiments 18-25, the method further comprising: heating the viscous material with a heater configured to heat the solution to a constant temperature to decrease the solution viscosity.

Embodiment 27: The method of Embodiment 26, wherein the heater comprises: a heating pad; and a metal tube surrounding the barrel mounted to the heating pad.

Embodiment 28: The method of Embodiment 27, wherein the heater further comprises a heating jacket surrounding the metal tube.

Embodiment 29: The method of any one of Embodiments 26-28, wherein the heater heats the viscous material to a temperature between 20 and 80 °C.

Embodiment 30: The method of any one of Embodiments 18-29, wherein the first valve and the second valve are a first solenoid and a second solenoid, respectively.

Embodiment 31: The method of any one of Embodiments 18-30, wherein the viscous material is a gel.

Embodiment 32: The method of any one of Embodiments 31, wherein the viscous material is a natural polymer-based gel, a synthetic polymer gel, or a natural-synthetic hybrid polymer gel.

Embodiment 33: The method of any one of Embodiments 18-32, wherein the viscous material comprises chitosan.

Embodiment 34: The method of Embodiment 33, wherein the viscous material further comprises alginate.

Embodiment 35: The method of any one of Embodiments 18-34, wherein the viscous material comprises chitosan-alginate gel.

Embodiment 36: The method of any one of Embodiments 18-33, wherein the viscous material comprises chitosan and hyaluronic hybrid gel.

Embodiment 37: The method of any one of Embodiments 18-36, wherein the viscous material comprises a polymer solution gel with encapsulated cells.

Embodiment 38: The method of any one of Embodiments 18-37, wherein the viscous material has a viscosity of about 8.9×10-4 Pa·s to 7000 Pa·s.

Embodiment 39: The method of any one of Embodiments 18-38, wherein the barrel is filled with one or more layers of two or more gels.

Embodiment 40: A cell scaffold made by any one of the methods of Embodiments 18-39, wherein the cell scaffold has a uniform volume.

Embodiment 41: The cell scaffold of Embodiment 40, wherein the cell scaffold has a uniform volume of 5% for a 96-well-plate.

Embodiment 42: The cell scaffold of Embodiment 40, wherein the cell scaffold has a uniform volume of 8% for a 384 well-plate

Embodiment 43: The cell scaffold of any one of Embodiments 40-42, wherein the cell scaffold is a porous cell scaffold.

Embodiment 44: The cell scaffold of Embodiment 43, wherein the porous cell scaffold has uniform porosity.

Embodiment 45: The cell scaffold of any of Embodiments 40-44, wherein the cell scaffold height is uniform among wells of a microplate.

Embodiment 46: The cell scaffold of Embodiment 45, wherein the cell scaffold has a uniform height of 5% for a 96 well plate.

Embodiment 47: The cell scaffold of Embodiment 45, wherein the cell scaffold has a uniform height of 8% for a 384 well plate.

Embodiment 48: An article of manufacture comprising a cell scaffold of any one of Embodiments 40-47.

Embodiment 49: The article of manufacture of Embodiment 48, wherein the article is a cell scaffold for 3D cell culture for fundamental biological studies, 3D in vitro tumor model for drug screening, 3D in vitro disease model for drug discovery, or 3D cell culture for primary and stem cell expansion.

1. A viscous material dispenser, comprising:
an extruder comprising an inlet, a barrel, and a nozzle;
a pump configured to fit inside the barrel;
a tube connected to the inlet, wherein the tube branches into at least two arms;
a first valve attached to a first arm of the tube, wherein the first valve opens and closes to a source of pressure; and
a second valve attached to a second arm of the tube,
wherein when the first valve is open and the second valve is closed, the pump is depressed by the source of pressurized air, causing a viscous material to flow out of the nozzle.

2. The viscous material dispenser of claim 1, wherein when the first valve is closed and the second valve is open, the viscous material stops flowing out of the nozzle.

3. The viscous material dispenser of claim 1, wherein the first valve, the second valve, or a combination thereof is selected from a pneumatic valve, a peristaltic valve, a piezoelectric valve, a hydraulic valve, an electromagnetic pump, or a progressive cavity valve.

4. The viscous material dispenser of claim 1, wherein the source of pressure is pressurized gas, pressurized liquid, or a combination thereof.

5. (canceled)

6. The viscous material dispenser claim 1, wherein the pump is a plunger.

7. The viscous material dispenser of claim 1, wherein the pump is a progressive cavity pump.

8. The viscous material dispenser of claim 1, wherein the viscous material dispenser further comprises a stepper-motor powered moveable stage configured to move a substrate horizontally and vertically in precise step sizes so that one well of the substrate is always directly beneath the nozzle of the extruder when the stepper-motor powered moveable stage is stationary.

9. The viscous material dispenser of claim 1, wherein the viscous material dispenser is multiplexed.

10. The viscous material dispenser of claim 1, wherein the second valve opens and closes to the atmosphere.

11. The viscous material dispenser of claim 1, wherein the viscous material dispenser further comprises a heater configured to heat the viscous material to a constant temperature to decrease the viscous material's viscosity.

12. The viscous material dispenser of claim 11, wherein the heater comprises:
a heating pad; and
a metal tube surrounding the barrel and mounted to the heating pad.

13. The viscous material dispenser of claim 12, wherein the metal tube is an aluminum tube.

14. The viscous material dispenser of claim 13, wherein the heater further comprises a heating jacket surrounding the metal tube.

15. The viscous material dispenser of claim 11, wherein the heater heats the viscous material to a temperature between 20 °C and 80 °C.

16. The viscous material dispenser of claim 11, wherein the first valve and the second valve are a first solenoid and a second solenoid, respectively.

17. The viscous material dispenser of claim 11, wherein the extruder is a syringe.

18. A method of dispensing a viscous material using a viscous material dispenser according to any of the preceding claims, the method comprising:
positioning a substrate underneath the nozzle;
filling the barrel of the extruder with a viscous material; and
dispensing the viscous material from the nozzle of the extruder and onto the substrate by opening the first valve and closing the second valve.

19-39. (canceled)

40. A cell scaffold made by the method of claim 18, wherein the cell scaffold has a uniform volume.

41-47. (canceled)

48. An article of manufacture comprising a cell scaffold of claim 40.

49. (canceled)

50. A viscous material dispenser, comprising:
an extruder comprising an inlet, a barrel, and a nozzle;
a pump configured to fit inside the barrel;
a tube connected to the inlet, wherein the tube branches into at least two arms;
a first valve attached to a first arm of the tube, wherein the first valve opens and closes to a source of pressure;
a stepper-motor powered moveable stage configured to move a substrate horizontally and vertically in precise step sizes so that one well of the substrate is always directly beneath the nozzle of the extruder when the stepper-motor powered moveable stage is stationary; and
a second valve attached to a second arm of the tube,
wherein when the first valve is open and the second valve is closed, the pump is depressed by the source of pressurized air, causing a viscous material to flow out of the nozzle.
